(19) United States
(12) Patent Application Publication
(10) Pub. No.: US 2022/0249467 A1
(43) Pub. Date: Aug. 11, 2022

(54) METHOD FOR IDENTIFYING TREATMENT OF INFECTIONS CAUSED BY PATHOGENS OF DIVERSE ORIGIN

(71) Applicant: SystaMedic Inc., Clinton, CT (US)

(72) Inventor: Anton Franz Joseph Fliri, Mystic, CT (US)

(73) Assignee: SystaMedic Inc., Clinton, CT (US)

(21) Appl. No.: 17/574,781

(22) Filed: Jan. 13, 2022

Related U.S. Application Data
(60) Provisional application No. 63/136,697, filed on Jan. 13, 2021.

(51) Int. Cl. A61K 31/496; A61K 31/167; A61K 31/122; A61K 31/4184; A61K 31/426; A61K 31/4406; A61K 31/155; A61K 31/047; A61K 31/203; A61K 31/325; A61K 31/223; A61P 31/14; C12N 15/10

(52) U.S. Cl. CPC A61K 31/496; A61K 31/167; A61K 31/122; A61K 31/4184; A61K 31/426; A61K 31/4406; C12N 15/1058; A61K 31/047; A61K 31/203; A61K 31/325; A61K 31/223; A61P 31/14; A61K 31/155

(57) ABSTRACT

The disclosed subject matter relates to the construction and use of novel proteomics-based descriptor sets for analyzing high throughput proteomics derived pathogen-host interactome information and the use of these descriptor sets for identifying substances and substance combinations that have utility for treating and or preventing infections and diseases caused by a broad range of pathogens of diverse origins.

### CROSS-REFERENCE TO RELATED APPLICATIONS

This application claims priority to and the benefit of U.S. Provisional Patent Application Ser. No. 63/136,697 filed Jan. 13, 2021, the disclosure of which is incorporated herein by reference in its entirety.

### FIELD

This disclosure relates to pharmaceutical compositions, pharmaceutical combinations and methods for treating and or preventing infections and diseases caused by a broad range of pathogens of diverse origins.

### BACKGROUND

Critical for developing countermeasures against pandemics is information on how pathogens affect cellular machinery and tissues of infected hosts. Infection in this context refers to an invasion of the body by harmful microorganisms or parasites. Among methods for securing this information are high throughput proteomics screening methodologies which are aimed at identifying how pathogens affect interactions between host proteins (this information is referred to as host-pathogen interactome). However, high throughput proteomics derived interactome information derived from different studies frequently shows very little overlap even in cases where methodologies used for ascertaining host pathogen interactions are similar (see e.g., L. Perfetto, C Pastrello, N del-Toro, M Duesbury, M lannuccelli, M Kotlyar, L Licata, B Meldal, K Panneerselvam, S Panni, N Rahimzadeh, S Ricard-Blum, L Salwinski, A Shrivastava, G Cesareni, M Pellegrini, S Orchard, I Jurisica, H Hermjakob, P Porras, The IMEx coronavirus interactome: an evolving map of Coronaviridae-host molecular interactions, Database, Volume 2020, 2020, baaa096 and Wodak S. J., Vlasblom J., Turinsky A. L. et al. (2013) Protein-protein interaction networks: the puzzling riches. Curr. Opin. Struct. Biol, 23, 941-953.). Causing uncertainty in assessing relevance of high throughput proteomics derived interactome information for development of counter measures against new pathogens, methodology that assists in interpreting pathogen associated high throughput proteomics derived screening data and in the identification of pandemic countermeasures has therefore utility. Accordingly, the aspects of the present disclosure described herein relate to the construction and use of novel proteomics-based descriptor sets for analyzing high throughput proteomics derived pathogen-host interactome information and the use of these descriptor sets for identifying substances and substance combinations that have utility for treating and or preventing infections caused by a broad range of pathogens of diverse origins.

### SUMMARY

In one embodiment, a combination of substances is provided. The combination of substances includes at least one compound selected from a first group of substances consisting of Niclosamide, Atovaquone, Posaconazole, Nocodazole, Nitazoxanide, JACOM Formulation, Kabasura Kudineer Chooranam preparations, Sura Kudineer preparations, Da Yuan Yin preparations, Lian Hua Qing Wen Capsule preparations, Ma Xin Gan Shi Tang preparations, Shuang Huang Lian preparations, Yin Qiao San preparations, Yu Ping Feng San preparation and combinations thereof and one or more compounds selected from a second group consisting of 4-hydroxy-2-nonenal, 5-Amino Levulinic Acid, 7-Ketocholesterol, Abemaciclib, Abiraterone, Acetaminophen, acetylcholine, Adavosertib, Afatinib, Alectinib, Alisertib, Alpelisib, Amlodipine, Amprenavir, Anisomycin, Aphidicolin, Arecolin, Artesunate, Aspirin, Astaxanthin, Auranofin, Axitinib, Baicalin, Berberine, Bermoprofen, Bevacizumab, Bexarotene, Bosentan, Bosutinib, Bromodomain Inhibitors, Bromodomain inhibitor JQ1, Buparlisib, Caduet, Caffeine, calcitriol, Candesartan, celastrol, Celecoxib, Ceritinib, Chloramphenicol, Cholecalciferol, CI-1040, Ciglitazone, Cilostazol, Clarithromycin, Colchicine, Copanlisib, Costunolide, Dabrafenib, Dacomitinib, Dicumarol, Dileucine methyl ester, Di-Leucine, Disulfiram, Dizocilpine, Docosahexaenoic Acid, Eicosapentaenoic acid, Eicosapentaenoic acid ethyl ester, Doxazosine, Duvelisib, Emodin, Enoxolone, Entinostat, Enzalutamide, Enzastaurin, Epoprostenol, Epoxyeicosatrienoic acid, Eribulin, Erlotinib, Evodiamine, Exemestane, Fasudil, Fedratinib, Fenofibrate, Fingolimod, Fluoxetine, Gedatolisib, Geldanamycin, Genistein, Givinostat, haloperidol, Hernandezine, Herring Roe Oil, Hymecromone, Icaritin, Icotinib, Idelalisib, Ilomastat, Imatinib, Indomethacin, irinotecan, Ixabepilone, Kaempferol, Krill Oil, Lapatinib, Lenalidomide, Lenvatinib, Letrozole, Liothyronine, Losartan, Lovastatin, Luminespib, LY294002, Medroxyprogesterone, Melatonin, Menadione, Metformin, Methotrexate, Myoinositol, Nebivolol, Nilotinib, Nimbolide, Niraparib, Obatoclax, Olaparib, Omeprazole, Ondansetron, Orlistat, Osimertinib, Osthol, oxonic acid, Palbociclib, Panobinostat, PD-0325901, Pemetrexed, Perifosine, Phenformin, Phenobarbital, piperine, plicamycin, Plitidepsin, Ponicidin, Pracinostat, Pristimerin, profolol, propofol, Pterostilbene, Puromycin, quercetin, Quinacrine, Raloxifene, Resveratrol, Retinal, Rhein, Ribociclib, Rosiglitazone, Rosuvastatin, Ruxolitinib, Salinomycine, Salvianolic acid, Saracatinib, Selumetinib, Semaxanib, Sildenafil, Simvastatin, SNX-2112, Sorafenib, SP600125, Sphingosine, STAT3 Inhibitor S3I-201, Suldinac, Sulforophane, Sunitinib, Tamoxifen, Tamsulosin, Taselisib, Telmisartan, Teprenone, Tetracycline, Tetrahydrocurcumin, Tetrandrine, Thalidomide, Thymoquinone, Tipifarnib, Transretinoicacid, Triamcinolone, Trichostatin A, Troglitazone, Trolox, Tyrphostin, Umbralisib, Ursolicacid, Veliparib, Venetoclax, Verapamil, Vinorelbine, Vitamin B12, Vorinostat, Vytorin, Withaferin A, pharmaceutically acceptable zinc salts, zinc acetate, zinc citrate, zinc gluconate, zinc pantothenate, Zinc sulfate, Zinc oxide, Zinc chloride, Zinc phosphate, and a pharmaceutically acceptable zinc complexes.

In another embodiment, a method for treatment or preventing infections in a mammal caused by pathogens selected from the group consisting of Bacterium Tuberculosis, Influenza Virus H7N2, Borna Disease Viruses, Body-1, Bodv-2, Influenza Virus H9N2, Chagas Disease, American Trypanosomiasis, Kaposi's Sarcoma-Associated Herpes virus, Human Coronavirus 229E, Hcov-229E, Lassa Virus, Crimean-Congo Hemorrhagic Fever, Leishmaniasis, Dengue Virus, Malaria, Ebola Virus, Marburg Virus, Endogenous Retroviruses, Measles, Epstein-Barr Virus, Nipah Virus, Escherichia Coli, Pertussis, Filovirus, Prion Diseases, Helicobacter Pylori, Respiratory Syncytial Virus, Hendra Henipavirus, Rift Valley Fever, Phlebovirus, Henipaviral Diseases, Salmonella, Hepatitis B Virus, Severe Acute Respiratory Syndrome Virus, Hepatitis C Virus, Shigellosis, Herpes Simplex Virus, Toxoplasmosis, HTLV-I Virus, Human Metapneumovirus, West Nile Virus, Human Papillomavirus Virus, Zika Virus, Influenza A Virus, Severe Acute Respiratory Syndrome Coronavirus SARS-Cov-2, Severe Acute Respiratory Syndrome Coronavirus SARS-Cov-1, Middle East Respiratory Syndrome Virus MERS, Trichophyton, Microsporum, Epidermophyton species, Candida, Aspergillus, Cryptococcus, and Pneumocystis. The method includes administering to said mammal in need of such treatment or prevention an effective amount of at least one compound selected from a first group of substances consisting of Niclosamide, Atovaquone, Posaconazole, Nocodazole, Nitazoxanide, JACOM Formulation, Kabasura Kudineer Chooranam preparations, Sura Kudineer preparations, Da Yuan Yin preparations, Lian Hua Qing Wen Capsule preparations, Ma Xin Gan Shi Tang preparations, Shuang Huang Lian preparations, Yin Qiao San preparations, Yu Ping Feng San preparation and combinations thereof and an effective amount of one or more compounds selected from a second group consisting of 4-hydroxy-2-nonenal, 5-Amino Levulinic Acid, 7-Ketocholesterol, Abemaciclib, Abiraterone, Acetaminophen, acetylcholine, Adavosertib, Afatinib, Alectinib, Alisertib, Alpelisib, Amlodipine, Amprenavir, Anisomycin, Aphidicolin, Arecolin, Artesunate, Aspirin, Astaxanthin, Auranofin, Axitinib, Baicalin, Berberine, Bermoprofen, Bevacizumab, Bexarotene, Bosentan, Bosutinib, Bromodomain Inhibitors, Bromodomain inhibitor JQ1, Buparlisib, Caduet, Caffeine, calcitriol, Candesartan, celastrol, Celecoxib, Ceritinib, Chloramphenicol, Cholecalciferol, CI-1040, Ciglitazone, Cilostazol, Clarithromycin, Colchicine, Copanlisib, Costunolide, Dabrafenib, Dacomitinib, Dicumarol, Dileucine methyl ester, Di-Leucine, Disulfiram, Dizocilpine, Docosahexaenoic Acid, Eicosapentaenoic acid, Eicosapentaenoic acid ethyl ester, Doxazosine, Duvelisib, Emodin, Enoxolone, Entinostat, Enzalutamide, Enzastaurin, Epoprostenol, Epoxyeicosatrienoic acid, Eribulin, Erlotinib, Evodiamine, Exemestane, Fasudil, Fedratinib, Fenofibrate, Fingolimod, Fluoxetine, Gedatolisib, Geldanamycin, Genistein, Givinostat, haloperidol, Hernandezine, Herring Roe Oil, Hymecromone, Icaritin, Icotinib, Idelalisib, Ilomastat, Imatinib, Indomethacin, irinotecan, Ixabepilone, Kaempferol, Krill Oil, Lapatinib, Lenalidomide, Lenvatinib, Letrozole, Liothyronine, Losartan, Lovastatin, Luminespib, LY294002, Medroxyprogesterone, Melatonin, Menadione, Metformin, Methotrexate, Myoinositol, Nebivolol, Nilotinib, Nimbolide, Niraparib, Obatoclax, Olaparib, Omeprazole, Ondansetron, Orlistat, Osimertinib, Osthol, oxonic acid, Palbociclib, Panobinostat, PD-0325901, Pemetrexed, Perifosine, Phenformin, Phenobarbital, piperine, plicamycin, Plitidepsin, Ponicidin, Pracinostat, Pristimerin, profolol, propofol, Pterostilbene, Puromycin, quercetin, Quinacrine, Raloxifene, Resveratrol, Retinal, Rhein, Ribociclib, Rosiglitazone, Rosuvastatin, Ruxolitinib, Salinomycine, Salvianolic acid, Saracatinib, Selumetinib, Semaxanib, Sildenafil, Simvastatin, SNX-2112, Sorafenib, SP600125, Sphingosine, STAT3 Inhibitor S3I-201, Suldinac, Sulforophane, Sunitinib, Tamoxifen, Tamsulosin, Taselisib, Telmisartan, Teprenone, Tetracycline, Tetrahydrocurcumin, Tetrandrine, Thalidomide, Thymoquinone, Tipifarnib, Transretinoicacid, Triamcinolone, Trichostatin A, Troglitazone, Trolox, Tyrphostin, Umbralisib, Ursolicacid, Veliparib, Venetoclax, Verapamil, Vinorelbine, Vitamin B12, Vorinostat, Vytorin, Withaferin A, pharmaceutically acceptable zinc salts, zinc acetate, zinc citrate, zinc gluconate, zinc pantothenate, Zinc sulfate, Zinc oxide, Zinc chloride, Zinc phosphate, and a pharmaceutically acceptable zinc complexes.

In another embodiment, a combination of substances is provided, the combination of substances includes niclosamide and one or more compounds selected from a second group consisting of bexarotene, Celecoxib, PD184352, Ciglitazone, Evodiamine, Fingolimod, geldanamycin, Obatoclax, Ondansetron, Perifosine, Phenformin, Ponicidin, Raloxifene, Sorafenib, and Troglitazone.

In another embodiment, a combination of substances is provided, the combination of substances includes at least one compound selected from a first group of substances consisting of Docosahexaenoic Acid, Eicosapentaenoic acid, Krill oil, Herring roe oil, Eicosapentaenoic acid ethyl ester, Eicosatrienoic acid, Eicosatrienoic acid ethyl ester, cannabidiol, hemp oil and combinations thereof and one or more compounds selected from a second group consisting of Myo-inositol, Dileucine, mung bean protein, Krill protein, Herring roe protein, Pterostilbene, and Caffeine.

In another embodiment, a biological structure-function constraint topological descriptor set is provided. The biological structure-function constraint topological descriptor set includes topological descriptor set termed 11KTSPDS.

In another embodiment, a method for constructing descriptor set 11KTSPDS is provided. The method includes a first step of selecting tissue specific expression data of protein encoding genes, a second step of using said selected genes for construction protein-protein interaction networks termed primary networks, a third step of using gene enrichment analysis for identifying protein network nodes of said primary protein protein interaction networks that co-occur in gene ontology based biological process networks termed secondary networks providing protein network fragments creating protein network overlaps between said primary and secondary networks, a fourth step of collecting said protein network fragments in an intermittent database, a fifth step of using said collected protein network fragments for selecting network fragments containing no more than ten network nodes associated with a false discovery rate of at least 0.001, a sixth step for associating said selected protein network fragments with registration codes identifying biological process network and tissue network of origin and a seventh step of collecting eleven thousand one hundred of said registration code associated protein network fragments providing descriptor set 11KTSPDS.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

All compounds included in embodiments of the present disclosure include the compound itself as well as pharmaceutically acceptable salts thereof.

Of the 400 emerging infectious diseases recorded since the 1940, infections caused by bacteria and rickettsia account for 54%, viral or prion pathogens for 25%, protozoa for 11%, fungi 6% and helminths for 3% of the infections (see https://www.ncbi.nlm.nih.gov/pmc/articles/PMC5960580/). Among these pathogens, RNA viruses—namely, HIV, influenza H1N1 and H5H1, severe acute respiratory syndrome coronavirus, Lassa virus, Ebola virus, and Middle East respiratory syndrome coronavirus Viral pathogens caused the most devastating impact on societies. Thus, RNA viruses have the ability to replicate in numerous host species and use their ability to rapidly mutate for evading host responses and diminishing efficacy of preventive vaccination measures. Producing on average, more than two new species in a year, RNA viruses will continue to pose a threat to humanity for years to come. Thus, as of December 2020, over 80 million people globally have become infected with a new strain of the severe acute respiratory syndrome coronavirus SARS-CoV2, causing more than 1.8 million deaths. Efforts for countering this pandemic called Covid-19, has resulted in massive scientific efforts attempting to create an understanding of the biology of the SARS-CoV2 coronavirus virus, the root causes for its infectivity and molecular mechanisms enabling this virus to evade host responses and steps involved in disease progression. In case of COVID 19, information on SARS CoV-2 host protein interactions are summarized in the COVID-19 Disease Map compiled by Ostaszewski M., Mazein A., Gillespie M. E. et al. in “(2020) COVID-19 Disease Map, building a computational repository of SARS-CoV-2 virus-host interaction mechanisms. Sci Data, 7, 136.3”.

To cause a disease, a pathogen must overcome host defenses which operate at many system levels. Among these defenses is the sophisticated innate immune system which detects and prevents the growth of harmful pathogens. This front-line defense system involves molecular pattern-triggered immunity and pathogen effector-triggered immunity which restrict pathogen attacks and terminate the growth of pathogens. This protective mechanism is conserved across species including plants. However, efficacies of defense mechanisms differ between individuals, species and tissues (see Jo, Eun-Kyeong. “Interplay between host and pathogen: Immune defense and beyond.” Experimental & molecular medicine 51.12 (2019): 1-3). For identifying functional relationships between biological processes affected by different pathogens in different tissues a methodology described in U.S. Pat. No. 11,120,346, the disclosure of which is incorporated herein by reference in its entirety, can be used for identifying tissues and biological processes affected by 44 pathogens with diverse origins listed in Table 1.

TABLE 1

| | |
|---|---|
| Bacterium Tuberculosis | influenza virus H7N2, |
| Borna disease viruses, BoDV-1, | influenza virus H9N2 |
| BoDV-2 | |
| Chagas disease, American | Kaposi's sarcoma-associated |
| trypanosomiasis | herpesvirus |
| Human coronavirus 229E, HCoV- | Lassa virus |
| 229E | |
| Crimean-Congo hemorrhagic fever | Leishmaniasis |
| Dengue virus | Malaria |
| Ebola virus | Marburg virus |
| Endogenous retroviruses | Measles |
| Epstein-Barr virus | Nipah virus |
| Escherichia coli infection | Pertussis |
| filovirus | Prion diseases |
| Helicobacter pylori infection | Respiratory syncytial virus |
| Hendra henipavirus | Rift Valley fever, Phlebovirus |
| henipaviral diseases | Salmonella infection |
| Hepatitis B virus | Severe Acute Respiratory |
| | Syndrome virus, SARS |
| Hepatitis C virus | Shigellosis |
| Herpes simplex virus | Toxoplasmosis |
| HTLV-I infection | Viral carcinogenesis |
| Human metapneumovirus | West Nile virus |
| Human papillomavirus virus | Zika |
| Influenza A virus | Severe acute respiratory |
| | syndrome coronavirus, SARS- |
| | CoV-2 |

For ascertaining host protein interaction information involved in infections caused by table 1 pathogens, the tissue specific expression of 20233 protein encoding genes provided in the protein atlas (See, Uhlén M., Fagerberg L., Hallström B. M. et al. (2015) Proteomics. Tissue-based map of the human proteome. Science, 347, 1260419) can be used. For ascertaining the protein network connectivity associated with tissue specific gene expression data, the string platform (See, Szklarczyk D, et al., STRING v11: protein-protein association networks with increased coverage, supporting functional discovery in genome-wide experimental datasets. Nucleic Acids Res. 2019 January; 47:D607-613) can be used. Tissue associated protein interaction networks provided by the string platform were compared by determining coinvestigation frequencies of protein encoding genes making up the various tissue associated protein interaction networks. Hierarchical clustering of accumulated cooccurrence frequency information enables identification of tissue specific and tissue non-specific protein interaction networks (called primary networks) which were associated with unique identifiers. Furthermore, for identifying biological processes (secondary networks) regulated by tissue specific interaction network (primary network) we used the String platform's gene enrichment analysis can be used. This gene enrichment analysis step enables identification of network fragments containing network nodes that co-occur in primary and secondary networks (network overlaps). Collecting eleven thousand one hundred network fragments each containing no more than ten network nodes associated with a false discovery rate of at least 0.001 and associating fragments with registration codes identifying biological process and tissue of origin provided a first topological (and biological structure-function constraint) descriptor set which for reference purposes is called 11KTSPDS.

The 11KTSPD descriptor set can then used for identifying tissue specific and tissue non-specific biological processes affected by infections caused pathogens listed in Table 1. For ascertaining this information, we used a methodology described in U.S. Pat. No. 11,120,346, Thus, the names of the 42 pathogens identified in table 1 were used for identifying information densities in the eleven thousand one hundred network fragments of the tissue associated descriptor set in >25 million Medline abstracts. These information density measurements are obtained by using the name of a pathogen, for determining co-occurrence frequency counts using the name of network nodes constituting a network fragment in the Medline database and summing up the counts for all network nodes in a network fragment (hereinafter called information density measurements). Collecting eleven thousand one hundred information density measurements for each of the 42 pathogens provides a similarity matrix. Hierarchical clustering of the resulting similarity matrix (11100 network fragments×42 pathogen names) identified that all pathogens identified in Table 1 affect interactions between 106 host proteins (Table 2a) and, in doing so, affect functions of biological processes networks overlapping with 144 protein network fragments (Table 2b). For identifying biological processes (secondary networks) affected by table 1 pathogens, the String platform's gene enrichment analysis can be used. Thus, entering the 106 proteins identified in Table 2a (primary network) into the string platform identified overlapping biological processes networks (secondary networks). Selecting 1403 network fragments each containing fewer than 50 network nodes co-occurring in the primary and in a secondary network and associating each of the 1403 network fragments with a registration code identifying the primary and secondary network of origin, can provide a second descriptor set which for reference purposes is called “PATH PP I” descriptor set.

TABLE 2a
106 Host proteins affected by Table 1 pathogens

ACPP, HRAS, CDKN1A, PTEN, BCL2L11, MAPK1, DRD5, SHE, ADM, IGF1, CDKN2A,
PTEN, BECN1, MAX, E2F1, SOD1, AGER, IKBKB, CFLAR, PTK2, BID, MET, EDN1,
SOD2, AGT, IMPACT, CTLA4, PTK2B, BMP2, MTOR, EGFR, TGFBR1, AKT1, INS,
CTNNB1, PTPN1, BPNT1, NEDD4L, EOMES, TNF, ARNTL, IRS1, CYCS, PTPN11,
CAD, NFKB1, EPO, TNFRSF1A, ATG5, JAK2, CYP24A1, RAG1, CALR, NP, FAS,
TNFSF11, ATM, JUN, DNMT1, RAG2, CASP8, NR3C1, FGF1, TP53, AXL, JUND,
DNMT3A, RB1, CAT, PIGF, FOS, BAD, KIT, DNMT3B, RELA, CCL5, PIGS, FYN,
BCAR1, LIF, DRD1, RIPK1, CD40, PIK3CA, GATA1, BCL2L1, MAP2K1, DRD3, SHC1,
CDC42, PLA2G7, GLUL, CDK4, PPARG, HDAC2, XIAP, CDK1, PLK1, GRB2,
HIF1A, ZEB2, VEGFA, VDR, VAV1, TSC1, TRAF6.

TABLE 2b
Biological processes affected by Table 1 pathogens

| AFFECTED HOST | NETWORK NODES IN PROTEIN NETWORK FRAGMENTS |
|---|---|
| PROCESS | AFFECTED BY TABLE 1 PATHIGENS |
| positive regulation of | AGER_AGT_EGFR_IL33_JAK2_MYD88_S100 |
| inflammatory | A9_SERPINE1_TNF_TNFSF11 |
| response | |
| regulation of stress- | AGER_AKT1_BMP2_EDN1_EGFR_FAS_HRA |
| activated MAPK | S_MAP2K1_PTK2B_PTPN1_RIPK1_TNF_TNF |
| cascade | SF11_TRAF6_VEGFA |
| regulation of JNK | AGER_AKT1_EDN1_EGFR_HRAS_PTK2B_P |
| cascade | TPN1_RIPK1_TNF_TNFSF11_TRAF6 |
| regulation of | AGER_BAD_CASP8_CD86_CDK6_CDKN2A_{—} |
| leukocyte | CREB1_CTLA4_CTNNB1_FOS_IL2RA_JUN_{—} |
| differentiation | MTOR_RB1_RIPK1_TNF_TNFSF1_TRAF6 |
| regulation of ERK1 | AGER_BMP2_CCL5_EGFR_EPO_HRAS_JUN |
| and ERK2 cascade | _MAP2K1_NRP1_PTK2B_PTPN1_PTPN11_S |
| | HC1_TNF_TNFSF11_VEGFA |
| positive regulation of | AGER_BMP2_CCL5_EGFR_EPO_HRAS_JUN |
| ERK1 and ERK2 | _MAP2K1_NRP1_PTK2B_PTPN11_SHC1_TN |
| cascade | F_TNFSF11_VEGFA |
| positive regulation of | AGER_CALR_EDN1_EGFR_RELA_TNF_TRF |
| NIK/NF-kappaB signaling | 6 |
| regulation of reactive | AGT_AKT1_BECN1_CDKN1A_EDN1_EGFR_{—} |
| oxygen species | FOXO3_FYN_GRB2_HIF1A_INS_JAK2_MTOR |
| metabolic process | _PTK2B_RIPK1_SHC1_TNF_TP53 |
| regulation of smooth | AGT_AKT1_CCL5_CDKN1A_CTNNB1_EDN1_{—} |
| muscle cell | EGFR_IGF1_JAK2_JUN_MTOR_PPARG_TNF |
| proliferation | _TRAF6 |
| positive regulation of | AGT_AKT1_CCL5_EDN1_EGFR_IGF1_JAK2_{—} |
| smooth muscle cell | JUN_MTOR_TNF_TRAF6 |
| proliferation | |
| positive regulation of | AGT_AKT1_CDKN1A_EDN1_EGFR_FOXO3_{—} |
| reactive oxygen | GRB2_JAK2_MTOR_PTK2B_RIPK1_TNF_TP5 |
| species metabolic | 3 |
| process | |
| regulation of | AGT_AKT1_EDN1_EGFR_HIF1A_INS_NFKB1 |
| oxidoreductase | _PTK2B_TNF |
| activity | |
| regulation of hormone | AGT_APOA1_ARNTL_BAD_BMP2_CCL5_CR |
| levels | EB1_EDN1_EGFR_HIF1A_INS_IRS1_JAK2_K |
| | CNJ11_NFKB1_PTPN11_RBP4_TNF_TNFSF1 |
| | 1 |
| regulation of hormone | AGT_ARNTL_BAD_CCL5_CREB1_EDN1_EG |
| secretion | FR_HIF1A_INS_IRS1_JAK2_KCNJ11_PTPN11 |
| | _RBP4_TNF_TNFSF11 |
| positive regulation of | AGT_BAD_CD274_CD58_CREB1_EDN1_EGF |
| secretion | R_HIF1A_IGF1_IL33_INS_JAK2_PTPN11_RB |
| | _P4_S100A9_TNF_TNFSF11_VEGFC |
| positive regulation of | AGT_BAD_CD274_CD58_CREB1_EDN1_EGF |
| secretion by cell | _R_HIF1A_IGF1_IL33_INS_JAK2_PTPN11_RB |
| | _P4_TNF_TNFSF11_VEGFC |
| negative regulation of | AGT_CDKN1A_CDKN2A_HIF1A_NRP1_PPAR |
| growth | G_PTK2_RBP4_SLIT2_TNF_TP53 |
| regulation of superoxide | AGT_EGFR_SHC1_TNF |
| metabolic process | |
| cellular response to | AKT1_ATG5_AXL_CYP24A1_GLUL_IMPACT_{—} |
| external stimulus | MAPK1_MAX_MTOR_SOD1_TNFRSF1A_VDR |
| response to radiation | AKT1_ATM_BCL2L1_CDKN1A_CHEK2_CREB |
| | 1_EGFR_FOS_GRB2_HIF1A_HRAS_JUN_KIT |
| | _MTOR_RELA_SAG_TP53 |
| response to reactive | AKT1_AXL_CAT_CFLAR_HDAC2_IMPACT_M |
| oxygen species | APK1_SOD1_SOD2 |
| cellular response to | AKT1_AXL_CFLAR_HDAC2_IMPACT_MAPK1 |
| reactive oxygen | _SOD1_SOD2 |
| species | |
| response to acid | AKT1_BAD_BCL2L1_CDK4_CFL1_COL1A2_C |
| chemical | REB1_E2F1_EDN1_EGFR_FYN_MTOR_PPA |
| | _RG_PTK2B_RBP4_RELA_TNF_VEGFA |
| positive regulation of | AKT1_BAD_BCL2L11_BID_BTRC_CASP8_CD |
| proteolysis | KN2A_FAS_FYN_IL33_JAK2_PLK1_PPARG_{—} |
| | PTK2_PTK2B_RB1_RIPK1_S100A9_TNF |
| regulation of cysteine- | AKT1_BAD_BCL2L11_BID_CASP8_CDKN2A_{—} |
| type endopeptidase | FAS_FYN_JAK2_PPARG_RIPK1_RPS6KA1_S |
| activity | 100A9_SOX2_TNF_VEGFA_XIAP |
| regulation of cysteine- | AKT1_BAD_BCL2L11_BID_CASP8_CDKN2A_{—} |
| type endopeptidase | FAS_JAK2_PPARG_RIPK1_RPS6KA1_S100A |
| activity involved in | 9_SOX2_TNF_VEGFA_XIAP |
| apoptotic process | |
| positive regulation of | AKT1_BAD_BID_CASP8_CDK1_CDKN2A_E2 |
| cellular protein | F1_EGFR_FYN_HRAS_IGF1_INS_JAK2_KCN |
| localization | J11_PLK1_TNF_TP53_VEGFA |
| epidermal growth | AKT1_BCAR1_EGFR_GRB2_PIK3CA_PTK2_{—} |
| factor receptor | PTK2B_PTPN11_SHC1 |
| signaling pathway | |
| negative regulation of | AKT1_BCL2L1_BECN1_CDKN2A_DFFA_E2F1 |
| cellular catabolic | _ELAVL1_INS_MET_MTOR_MYD88_PIK3CA_{—} |
| process | PTK2_TIMP2_TSC1 |
| cellular response to | AKT1_BCL2L1_CDK4_COL1A2_CREB1_E2F1_EDN1_EGFR_FYN_MTOR_PPARG_PTK2B |
| acid chemical | TNF_VEGFA |
| regulation of protein | AKT1_CD86_EGFR_FGF1_FYN_GRB2_INS_I |
| kinase B signaling | RS1_KIT_KL_MET_MTOR_PIK3CA_PTK2_PT |
| | PN11_TGFBR1_TNF_TNFSF11_VAV1 |
| regulation of | AKT1_EGFR_HIF1A_INS_NFKB1_PTK2B_TN |
| monooxygenase | F |
| activity | |
| positive regulation of | AKT1_EGFR_PIK3CA_TNF_VEGFA |
| peptidyl-serine | |
| phosphorylation | |
| negative regulation of | APOA1_ATM_CD274_CDK6_CDKN2A_CTLA4 |
| immune system | _CTNNB1_IL2RA_IL33_INS_PPARG_PTK2B_{—} |
| process | SLIT2_TNF |
| glycerophospholipid | APOA1_ATM_CD86_EGFR_FGF1_FYN_GRB |
| metabolic process | 2_IRS1_KIT_KL_MET_PIK3CA_PTPN11_VAV |
| | 1 |
| regulation of peptide | ARNTL_BAD_CCL5_EGFR_HIF1A_INS_IRS1_{—} |
| hormone secretion | JAK2_KCNJ11_PTPN11_RBP4_TNF_TNFSF1 |
| | 1 |
| response to starvation | ATG5_CAD_GLUL_IMPACT_MAPK1_MAX_M |
| | TOR |
| cellular response to | ATG5_CYP24A1_GLUL_IMPACT_MAPK1_MA |
| nutrient levels | X_MTOR_SOD1_VDR |
| cellular response to | ATG5_GLUL_IMPACT_MAPK1_MAX_MTOR |
| starvation | |
| apoptotic signaling | ATM_BAD_BCL2L1_BCL2L11_BID_CASP8_C |
| pathway | D40_CDKN1A_CHEK2_E2F1_FAS_FOXO3_H |
| | RAS_IL33_JAK2_JUN_RIPK1_TNF_TP53 |
| intrinsic apoptotic | ATM_BAD_BCL2L1_BCL2L11_CDKN1A_CHE |
| signaling pathway | K2_E2F1_HRAS_JAK2_TNF_TP53 |
| intrinsic apoptotic | ATM_BAD_BCL2L1_BCL2L11_CDKN1A_CHE |
| signaling pathway in | K2_E2F1_TNF_TP53 |
| response to DNA | |
| damage | |
| negative regulation of | ATM_BCL2L1_CDK1_CDK2_CDKN1A_CHEK2 |
| mitotic cell cycle | _CTNNB1_E2F1_EGFR_EZH2_HRAS_MDM4 |
| | _PLK1_RB1_TIMP2_TNF_TP53 |
| lipid modification | ATM_CD86_EGFR_FGF1_FYN_GRB2_IRS1_{—} |
| | KIT_KL_MET_PIK3CA_PPARG_PTPN11_VAV |
| | 1 |
| phosphatidylinositol | ATM_CD86_EGFR_FGF1_FYN_GRB2_IRS1_{—} |
| phosphorylation | KIT_KL_MET_PIK3CA_PTPN11_VAV1 |
| positive regulation of | ATR_CD4_CDK1_FOXP3_FYN_MALT1_RC3H |
| cellular metabolic | 1 |
| process | |
| positive regulation of | ATR_CD4_CDK1_FOXP3_FYN_MALT1_RC3H |
| macromolecule metabolic | 1 |
| process | |
| positive regulation of | ATR_CD4_CDK1_FOXP3_RC3H1 |
| nucleobase-containing | |
| compound metabolic | |
| process | |
| positive regulation of | ATR_CD4_CDK1_FYN_IL7_MALT1_RC3H1 |
| signal transduction | |
| positive regulation of | ATR_CD4_CDK1_FYN_MALT1_RC3H1 |
| intracellular signal | |
| transduction | |
| cellular response to | AXL_HDAC2_IMPACT |
| hydrogen peroxide | |
| interaction with host | BAD_BCL2L1_BCL2L11_CASP8_CD86_CDK1 |
| | _CTNNB1_EGFR_GRB2_ITGB3_MET |
| response to amino acid | BAD_BCL2L1_CFL1_COL1A2_CREB1_EDN1_{—} |
| | EGFR_FYN_MTOR_RELA_TNF |
| positive regulation of | BAD_BCL2L11_BID_CASP8_CDKN2A_FAS_F |
| cysteine-type | YN_JAK2_PPARG_RIPK1_S100A9_TNF |
| endopeptidase activity | |
| response to | BAD_BCL2L11_CDKN1A_EDN1_EGFR_EIF4E |
| glucocorticoid | BP1_EPO_FOS_FOXO3_SLIT2_TNF |
| cellular response to drug | BAD_BECN1_CDK1_CDK2_CDK4_CHEK2_C |
| | TNNB1_EDN1_EGFR_EIF4EBP1_EZH2_FYN_{—} |
| | KCNJ11_NFKB1_RELA_SLIT2_TNF_TP53 |
| positive regulation of | BAD_BID_CASP8_CD274_CD58_CDK1_E2F1 |
| establishment of protein | _EGFR_FYN_HIF1A_HRAS_IGF1_IL33_INS_J |
| localization | AK2_RBP4_TNF_TP53_VEGFC |
| positive regulation of | BAD_CD274_CD58_CDK1_EGFR_FYN_HIF1 |
| protein transport | A_HRAS_IGF1_IL33_INS_JAK2_RBP4_TNF_T |
| | P53_VEGFC |
| positive regulation of | BAD_CD274_CD58_EGFR_HIF1A_IGF1_IL33 |
| peptide secretion | _INS_JAK2_RBP4_S100A9_TNF_TNFSF11_V |
| | EGFC |
| positive regulation of | BAD_CD274_CD58_EGFR_HIF1A_IGF1_IL33 |
| protein secretion | _INS_JAK2_RBP4_TNF_VEGFC |
| cellular response to | BCL2L1_COL1A2_EGFR_FYN_MTOR_TNF |
| amino acid stimulus | |
| positive regulation of cell | BMP7_CD46_CSF2_CX3CR1_IL23A_MAP2K1 |
| differentiation | _POR_TGFB1_TGFBR1_THPO_TLR2_TNFSF |
| | 11_TNFSF4 |
| gland morphogenesis | BTRC_CAPN1_EGFR_NRP1_RPS6KA1_TNF |
| response to starvation | CAD_GLUL_IMPACT_MAX |
| defense response to | CAMP_CCL20_CD207_CD40_CFP_CLEC7A_{—} |
| other organism | HRAS_IL23A_MYD88_PCBP2_RAG2_TGFB1_{—} |
| | TLR2_TLR3_TLR8_TNFRSF1A_TNFSF4_TRI |
| | M5_TSLP |
| regulation of cytokine | CCL20_CCR7_CD40_CD46_CSF2_CYBB_HR |
| production | AS_IL23A_MYD88_PRG2_PRNP_RELB_TGF |
| | B1_TLR2_TLR4_TNFSF4_TSLP |
| cellular response to | CD4_CD69_CDK1_FYN_IL7_RC3H1 |
| chemical stimulus | |
| viral entry into host cell | CD4_CDK1 |
| regulation of multicellular | CD4_CDK1_CTLA4_FOXP3_FYN_IL7_MALT1 |
| organismal process | _PDCD1LG2_PNP_RC3H1 |
| regulation of cell | CD4_CDK1_CTLA4_FOXP3_FYN_IL7_MALT1 |
| differentiation | _PNP_RC3H1 |
| regulation of multicellular | CD4_CDK1_CTLA4_FOXP3_FYN_IL7_MALT1 |
| organismal development | _PNP_RC3H1 |
| regulation of cell | CD4_CDK1_CTLA4_FOXP3_FYN_IL7_PDCD1 |
| population proliferation | LG2_PNP_RC3H1 |
| positive regulation of | CD4_CDK1_FOXP3_FYN_IL7_MALT1_PNP |
| developmental process | |
| positive regulation of | CD4_CDK1_FOXP3_FYN_IL7_{——}MALT1_PNP |
| multicellular organismal | |
| process | |
| cell differentiation | CD4_CDK1_FOXP3_FYN_IL7_MALT1_RC3H1 |
| positive regulation of | CD4_CDK1_FOXP3_FYN_MALT1 |
| protein modification | |
| process | |
| regulation of cellular | CD4_CDK1_FOXP3_FYN_MALT1_RC3H1 |
| protein metabolic | |
| process | |
| multi-organism process | CD4_CDK1_FOXP3_FYN_MALT1_TOP1 |
| positive regulation of cell | CD4_CDK1_FOXP3_IL7_PDCD1LG2_PNP |
| population proliferation | |
| response to organic | CD4_CDK1_FYN_IL7_MALT1_RC3H1 |
| substance | |
| positive regulation of | CD4_CDK1_FYN_MALT1 |
| protein kinase activity | |
| viral process | CD4_CDK1_FYN_TOP1 |
| adaptive immune | CD4_CTLA4_FOXP3_FYN |
| response | |
| positive regulation of T | CD4_CTLA4_FOXP3_FYN_IL7_MALT1_PDCD |
| cell activation | 1LG2_PNP |
| immune response | CD4_CTLA4_FOXP3_FYN_IL7_MALT1_PDCD |
| | 1LG2_PNP_RC3H1 |
| positive regulation of | CD4_CTLA4_FOXP3_FYN_IL7_MALT1_PDCD |
| immune system process | 1LG2_PNP_{—} |
| | RC3H1 |
| regulation of leukocyte | CD4_CTLA4_FOXP3_FYN_IL7_MALT1_PDCD |
| cell-cell adhesion | 1LG2_PNP_RC3H1 |
| regulation of T cell | CD4_CTLA4_FOXP3_FYN_IL7_MALT1 |
| activation | PDCD_1LG2_PNP_RC3H1 |
| cell surface receptor | CD4_CTLA4_FOXP3_FYN_IL7_MALT1_RC3H |
| signaling pathway | 1 |
| antigen receptor- | CD4_CTLA4_FOXP3_FYN_MALT1_RC3H1 |
| mediated signaling | |
| pathway | |
| regulation of leukocyte | CD4_CTLA4_FOXP3_IL7_MALT1_PNP_RC3H |
| differentiation | 1 |
| regulation of lymphocyte | CD4_CTLA4_FOXP3_IL7_PDCD1LG2_PNP_R |
| proliferation | C3H1 |
| regulation of T cell | CD4_CTLA4_FOXP3_PDCD1LG2_PNP_RC3H |
| proliferation | 1 |
| regulation of interleukin-2 | CD4_FOXP3 |
| biosynthetic process | |
| T cell selection | CD4_FOXP3 |
| positive regulation of cell | CD4_FOXP3_FYN_IL7_MALT1_PNP |
| differentiation | |
| leukocyte activation | CD4_FOXP3_FYN_IL7_MALT1_PNP_RC3H1 |
| T cell activation | CD4_FOXP3_FYN_IL7_MALT1_RC3H1 |
| T cell receptor signaling | CD4_FOXP3_FYN_MALT1_RC3H1 |
| pathway | |
| positive regulation of | CD4_FOXP3_IL7_MALT1 |
| cytokine production | |
| regulation of cytokine | CD4_FOXP3_IL7_MALT1_PDCD1LG2 |
| production | |
| positive regulation of | CD4_FOXP3_IL7_MALT1_PNP |
| leukocyte differentiation | |
| lymphocyte differentiation | CD4_FOXP3_IL7_MALT1_RC3H1 |
| positive regulation of | CD4_FOXP3_IL7_PDCD1LG2_PNP |
| lymphocyte proliferation | |
| T cell differentiation | CD4_FOXP3_IL7_RC3H1 |
| positive regulation of | CD4_FOXP3_MALT1 |
| adaptive immune | |
| response based on | |
| somatic recombination of | |
| immune receptors built | |
| from immunoglobulin | |
| superfamily domains | |
| regulation of interleukin-2 | CD4_FOXP3_MALT1 |
| production | |
| regulation of adaptive | CD4_FOXP3_MALT1_RC3H1 |
| immune response based | |
| on somatic | |
| recombination of immune | |
| receptors built from | |
| immunoglobulin | |
| superfamily domains | |
| positive regulation of T | CD4_FOXP3_PDCD1LG2_PNP |
| cell proliferation | |
| cytokine production | CD4_FOXP3_PNP |
| positive regulation of I- | CD4_FYN |
| kappaB kinase/NF- | |
| kappaB signaling | |
| positive regulation of | CD4_FYN |
| peptidyl-tyrosine | |
| phosphorylation | |
| regulation of calcium ion | CD4_FYN |
| transport into cytosol | |
| regulation of defense | CD4_FYN |
| response to virus by virus | |
| response to nutrient | CD4_FYN |
| cytokine-mediated | CD4_FYN_IL7 |
| signaling pathway | |
| cellular response to | CD4_FYN_IL7_RC3H1 |
| cytokine stimulus | |
| positive regulation of | CD4_MALT1 |
| interleukin-2 production | |
| positive regulation of | CD86_EGFR_FGF1_FYN_GRB2_INS_IRS1_K |
| protein kinase B | IT_KL_MET_MTOR_PIK3CA_PTK2_PTPN11_{—} |
| signaling | TGFBR1_TNF_TNFSF11_VAV1 |
| positive regulation of | CDK4_IMPACT_MAPK1_MTOR |
| translation | |
| positive regulation of | CDK4_IMPACT_MAPK1_MTOR_TNFRSF1A |
| cellular amide metabolic | |
| process | |
| cell cycle arrest | CDKN1B_CDKN2A_DDIT3_IL12A_IRF1_MYC |
| | _NBN_PML |
| cell cycle arrest | CDKN1B_CDKN2A_DDIT3_IL12A_IRF1_MYC |
| | _NBN_PML |
| positive regulation of | CDKN2A_CEBPA_FADD_MEFV_MYC_MYH9_{—} |
| proteolysis | NLRP3_PML_S100A8_S100A9_SIRT1_STUB1 |
| regulation of cysteine- | CDKN2A_FADD_GPX1_KLF4_LTF_MEFV_MY |
| type endopeptidase | C_NLRP3_PML_S100A8_S100A9_SIRT1 |
| activity | |
| regulation of cysteine- | CDKN2A_FADD_GPX1_KLF4_MYC_NLRP3_P |
| type endopeptidase | ML_S100A8_S100A9_SIRT1 |
| activity involved in | |
| apoptotic process | |
| positive regulation of | CDKN2A_FADD_MEFV_MYC_NLRP3_PML_S |
| cysteine-type | 100A8_S100A9_SIRT1 |
| endopeptidase activity | |
| positive regulation of | CDKN2A_FADD_MEFV_MYC_NLRP3_PML_S |
| cysteine-type | 100A8_S100A9_SIRT1 |
| endopeptidase activity | |
| positive regulation of | CDKN2A_FADD_MYC_NLRP3_PML_S100A8_{—} |
| cysteine-type | S100A9_SIRT1 |
| endopeptidase activity | |
| involved in apoptotic | |
| process | |
| positive regulation of | CDKN2A_FADD_MYC_NLRP3_PML_S100A8_{—} |
| cysteine-type | S100A9_SIRT1 |
| endopeptidase activity | |
| involved in apoptotic | |
| process | |
| positive regulation of | CFLAR_DNMT3B_IMPACT_MTOR_NEDD4L_{—} |
| neuron differentiation | PTEN_ZEB2 |
| vitamin D metabolic | CYP24A1_CYP27B1_GC_VDR |
| process | |
| cellular response to | DNMT1_DNMT3A_DRD1_DRD5_IMPACT_MA |
| organonitrogen | X_SOD1 |
| compound | |
| negative regulation of | DNMT1_DNMT3B_MET_MOS_SET_WAS |
| organelle organization | |
| response to osmotic | EGFR_EPO_MT-CYB_MYLK_PTK2B_TNF |
| stress | |
| peptidyl-tyrosine | EGFR_FGF1_FYN_GRB2_JAK2_KIT_MAP2K1 |
| phosphorylation | _MET_NRP1_PRLR_PTK2_PTK2B |
| salivary gland | EGFR_NRP1_TNF |
| morphogenesis | |
| cellular response to | IMPACT_MAPK1_MTOR |
| amino acid starvation | |
| cellular response to | IMPACT_MTOR |
| leucine starvation | |
| positive regulation of | IMPACT_MTOR |
| translational initiation | |
| regulation of actin | MET_MTOR_NF2_SMAD3_SPTB_WAS |
| filament organization | |
| positive regulation of | MET_MTOR_NF2_SMAD3_WAS |
| supramolecular fiber | |
| organization | |
| regulation of stress fiber | MET_MTOR_NF2_SMAD3_WAS |
| assembly | |
| negative regulation of | MET_WAS |
| stress fiber assembly | |

The PATHPP descriptor set can be useful for identifying functional relationships between 1366 SARS COV-2 interactome proteins identified in supplementary Table 5 of a data base constructed by Ostaszewski M., Mazein A., Gillespie M. E. et al. (2020) COVID-19 Disease Map, building a computational repository of SARS-CoV-2 virus-host interaction mechanisms. Sci Data, 7, 136.3.). Thus, for identifying functional relationships between SARS CoV-2 interactome proteins the PATHPPI descriptor set and the methodology described in U.S. Pat. No. 11,120,34627 can be used for determining information densities of the 1366 SARS CoV-2 interactome proteins in >25 million Medline abstracts using the names of network nodes constituting each one of the 1403 protein network fragments of the PATHPPI descriptor set. Again these information density measurements are obtained by using the name of a protein constituting the SARS CoV-2 interactome, for determining co-occurrence frequency counts using the name of network nodes constituting a network fragment of the PATHPPI descriptor set in the Medline daabase and summing up the counts for all network nodes in a network fragment. Collecting 1368 measurements for 1403 network fragments of the PATHPPI descriptor set can provide a similarity matrix. Hierarchical clustering of the resulting similarity matrix includes 1403×1368 information density measurements partitioned the 1366 SARS CoC-2 interactome proteins into 23 discrete subgroups. Proteins in each subgroup can be entered into the String platform and subjected to the Sting platform's gene enrichment analysis. The result of this PATHPPI enabled SARS CoV-2 interactome analysis is shown in Table 4. Inspection of biological processes affected by the 23 SARS Cov-2 interactome groupings identifies that SARS CoV-2 infections primarily impact on processes involved in blood coagulation, the regulation of innate immune system and TGF beta signaling. Thus, viewing SARS CoV-2 interactome information from the perspective of information provided by interactomes of 42 pathogens directly can identify the impact of SARS CoV-2 infections on host physiological functions. This therapeutic relevant information is not recognized by pathway centered interactome analysis provided for example, by Gordon, D. E., Jang, G. M., Bouhaddou, M. et al. A SARS-CoV-2 protein interaction map reveals targets for drug repurposing. Nature 583, 459-468 (2020). Accordingly, descriptor sets of the present disclosure can be useful for ascertaining functional relationships between proteins identified as pathogen specific interactomes.

TABLE 4

| | Number of |
|---|---|
| | biological |
| Functions affected by discrete groups of SARS CoV2 | processes |
| interactome proteins | affected |
| NOTCH pathway regulation of fibrinolysis/coagulation | 480 |
| TGFB-Activin signaling | 310 |
| innate immunity/virus host interactions | 212 |
| TP53-signaling cell cycle | 180 |
| RHO-RAB | 145 |
| NOTCH-ribosome | 75 |
| Wint-Ephrine signaling | 63 |
| endosome-protein transport | 46 |
| Fibrinolysis | 36 |
| Vesicular transport | 35 |
| Autophagy | 33 |
| Endocytosis | 10 |
| Viral RNA transport | 9 |
| ER-post translational modifications | 9 |
| ER-Golgi | 8 |
| Exocytosis | 8 |
| Platelet Degranulation | 8 |
| Viral Entry | 6 |
| EGFR-signaling | 4 |
| TP53-regulation of acetylation | 4 |
| Redox | 3 |
| IGF signaling | 2 |
| ATP production citrate cycle | 1 |

For identifying substances capable of affecting replication cycles of SARS CoV-2 corona virus, the biological process fragments affected by the 23 groupings of SARS CoV-2 interactome proteins can be collected in an intermittent database and associated with registration codes identifying biological processes and SARS CoV-2 interactome sub-network of origin. A selection of 1748 network fragments containing less than 50 network nodes overlapping with anyone of 23 SARS CoV2 interactome subnetworks (primary networks) and biological processes (secondary networks) can provide a third descriptor set which for reference purposes is called SC2ISD.

Likewise, for identifying substances capable of affecting replication cycles of influenza A virus, the curated influenza virus A interactome published in the KEgg database and consisting of 158 proteins (primary network) can be entered into the string platform and subjected to gene enrichment analysis. This analysis step identified host biological processes (secondary networks) overlapping with the Influenza A virus interactome and hence indicating susceptibility of a host biological process to modulation by the influence virus interactome. This analysis step allows selection of 766 Network fragments containing network nodes co-occurring in primary and secondary networks. These 766 Network fragments can be collected and associated with registration codes identifying biological process and network of origin. The collection of 766 influenza A interactome derived network fragments identifying overlaps between primary and secondary networks provides a fourth descriptor set which for reference purposes is called INFADS.

For identifying substances capable of affecting replication cycles of Table 1 pathogens, which includes Influenzas and corona viruses the PATHPPI, SC2ISD, INFADS descriptor sets and methodology can be used as described in U.S. Pat. No. 11,120,34627 for identifying biological processes constituting the PATH PPI, SC2ISD, INFADS descriptor sets affected by Table 5 medicines can have broad spectrum anti-infective properties. This protein network overlap analysis involved determination of information density measurements using the names of ingredients constituting a Table 5 medicine with network nodes constituting network fragments of the PATHPPI, SC2ISD, INFADS descriptor sets combining said measurements for constructing a similarity matrix followed by hierarchical clustering of the similarity matrix. This process identified that 149 proteins listed in Table 6 creating overlap between PATHPPI, SC2ISD, INFADS descriptor sets and that can be targeted by Table 5 medicines.

TABLE 5

| Name | Ingredients |
|---|---|
| Da Yuan Yin | Areca Catechu, Arecae Semen, |
| | Magnoliae Officinalis Cortex, Magnoliae |
| | Officinalis, Amomum Tsao-Ko, |
| | Anemarrhena Asphodeloides, Dioscorea |
| | Opposita, Scutellaria Baicalensis, |
| | Glycyrrhizae Uralensis, |
| Lian Hua Qing Wen Capsule | Forsythia Suspensa, Ephedra Sinica, |
| | Lonicera Japonica, Isatis Indigotica, |
| | Mentha Haplocalyx, Dryopteris |
| | Crassirhizoma, Rhodiola Rosea, Gypsum |
| | Fibrosum, Pogostemon Cablin, Rheum |
| | Palmatum, Houttuynia Cordata, |
| | Glycyrrhizae Uralensis, Armeniaca |
| | Sibirica, |
| Ma Xin Gan Shi Tang | Ephedra Sinica, Armeniacae |
| | Semenamarum, Glycyrrhizae Uralensis, |
| | Gypsum Fibrosum, Areca Catechu, |
| | Arecae Semen, Magnoliae Officinalis |
| | Cortex, Magnoliae Officinalis, Amomum |
| | Tsao-Ko, Anemarrhena Asphodeloides, |
| | Dioscorea Opposita, Scutellaria |
| | Baicalensis, Glycyrrhizae Uralensis, |
| Shuang Huang Lian | Lonicera Japonica, Scutellaria |
| | Baicalensis, Forsythia Suspensa, |
| Yin Qiao San | Fructus Forsythiae, Forsythia Suspensa, |
| | Flos Lonicerae, Lonicera Japonica, Radix |
| | Platycodonis, Platycodon Grandiflorum, |
| | Mentha Spicata, Metha Piperita, |
| | Lophatherum Gracile, Glycyrrhiza |
| | Uralensis, Radix Glycyrrhizae, |
| | Schizonepeta, Herba Schizonepetae, |
| | Fermented Soybean, Fructus Arctii, |
| | Arctium Lappa, Rhizoma Phragmitis, |
| | Phragmites Communis, |
| Yu Ping Feng San | Astragalus Propinquus, Astragalus |
| | Membranaceus, Atractylodes |
| | Macrocephala, Bai Zhu, Atractylodes |
| | Macrocephala, Saposhnikoviae Radix, |
| | Saposhnikovia Divaricata, |
| critical Cov19 | Panax Ginseng, Aconitum Carmichaelii, |
| mild CoV19 | Pogostemon Cablin, Atractylodes Lancea, |
| | Scutellaria Baicalensis, Bupleurum |
| | Chinense, Forsythia Suspensa, |
| moderate Cov19 | Gypsum Fibrosum, Atractylodes Lancea, |
| | Polygonum Cuspidatum, Pogostemon |
| | Cablin, Verbena Officinalis, |
| recovery Cov19 | Hedysarum Multijugum, Ophiopogon |
| | Japonicus, Panax Quinquefolius, |
| severe Cov19 | Ephedra Sinica, Gypsum Fibrosum, |
| | Descurainia Sophia, Lepidium Apetalum, |
| | Bufalo Horn, |
| JACOM_Formulation | Justicia Adathoda, Andrographis |
| | Paniculata, Ocimum Tenuiflorum, Melia |
| | Azedarach, |
| Kabasura_Kudineer_Chooranam | Zingiber Officinale, Piper Longum, |
| | Syzygium Aromaticum, Tragia |
| | Involucrata, Anacyclus Pyrethrum, |
| | Andrographis Paniculata, Hygrophila |
| | Auriculata, Terminalia Chebula, Justicia |
| | Adhatoda, Plectranthus Amboinicus, |
| | Costus Speciosus, Tinospora Cordifolia, |
| | Clerodendrum Serratum, Sida Acuta, |
| | Cypreus Rotundus, |
| Sura_Kudineer | Zingiber Officinale, Piper Longum, |
| | Syzygium Aromaticum, Anacyclus |
| | Pyrethrum, Tragia Involucrata, Tragus |
| | Involucrate, Hygrophila Auriculata, |
| | Terminalia Chebula, Justicia Adhatoda, |
| | Anisochilus Carnosus, Costus Speciosus, |
| | Cheilocostus Speciosus, Tinospora |
| | Cordifolia, Clerodendrum Serratum, |
| | Andrographis Paniculata, Cyperus |
| | Rotundus, Sida Acuta, |
| Niclosamide | |

References:
Xu, Jimin et al. “Broad Spectrum Antiviral Agent Niclosamide and Its Therapeutic Potential.” ACS infectious diseases vol. 6, 5 (2020): 909-915; Mohammad, Haroon et al. “Repurposing niclosamide for intestinal decolonization of vancomycin-resistant enterococci.” International journal of antimicrobial agents vol. 51, 6 (2018): 897-904.
Yang, Yang et al. “Traditional Chinese Medicine in the Treatment of Patients Infected with 2019-New Coronavirus (SARS-CoV-2): A Review and Perspective.” International journal of biological sciences vol. 16, 10 1708-1717. 15 Mar. 2020, doi:10.7150/ijbs.45538; Luo, H., Gao, Y., Zou, J. et al. Reflections on treatment of COVID-19 with traditional Chinese medicine. Chin Med 15, 94 (2020.
Kiran, Gangarapu et al., In Silico computational screening of Kabasura Kudineer - Official Siddha Formulation and JACOM against SARS-CoV-2 spike protein. Journal of Ayurveda and Integrative Medicine (2020)

TABLE 6
Protein network nodes (hereinafter called 146TNTW) connecting biological
processes affected by traditional medicines shown in Table 5

ABCC1, ABHD5, ACADM, ACLY, ACO1, ACSL3, ACVR2A, ADCY9, AP2A2,
AP2M1, APOB, AREG, ARFGEF2, ATG5, ATG9A, ATP1B1, ATP5D, ATP7B,
AXL, B2M, BAD, BCL1, BCL2L11, BECN1, BLVRA, C1QBP, CALM3, CASP8,
CAT, CCL5, CCT3, CCT4, CCT5, CCT6A, CCT7, CDC42, CDK1, CEP250,
CFLAR, CKAP4, CNTRL, COMT, COPG2, CORO1C, CRKL, CTDNEP1,
CTNNB1, CTSB, CUX1, DCAF7, DLD, DNAJA3, DRAM2, ECE1, EEA1, EGFR,
ELOVL7, ERBB2, EZR, FADS2, FGFR1, FH, FYN, GABARAPL2, GCNT3,
GGH, GOLGB1, GORASP2, GOSR2, GPAA1, GPX1, GRB2, HARS2, HOOK1,
HSPA5, IPO8, JAK2, JUN, KIAA0319, KIF5B, LDLR, LRP8, MAPK1, MET,
MFGE8, MT-ATP6, MT-CO2, MTHFD1L, MUL1, NAPG, NBR1, NEDD4L,
NPC2, NRG1, OAT, OS9, PIGO, PIGS, PIGT, PITRM1, PSMD1, PSMD11,
PTPN11, RAB21, RAB2A, RAB31, RAB5A, RAB5C, RAB7A, RAB9A, RANBP6,
RB1, RELA, RIPK1, RNF149, RTN4, SCAMP3, SEC61B, SEC61G, SHC1,
SIGMAR1, SLC30A6, SLC30A9, SLC9A3R1, SNAP25, SNX1, SNX2, SOD1,
SORT1, SPG11, SPNS1, STAM2, STRA6, STX12, SUMF2, TGOLN2,
TMEM97, TNFRSF1A, TOR1A, TP53, TRIM16, USP8, VIMP, VMP1, VPS11,
VPS16, VPS39, VPS41, WFS1.

a. Entering Table 6 proteins (primary network) into the String platform for gene enrichment analysis identifies biological processes (secondary networks) affected by Table 5 medicines. Selecting seven hundred seventeen network fragments containing less than 50 protein network nodes co-occurring in primary and secondary networks and associated with a false discovery rate of at least 0.001 followed by assignment of network fragment registration codes identifying biological process and primary network of origin provided a fifth descriptor set which for reference purposes is called 717P146TNTW.

TABLE 7
TTOP BIOLOGICAL PROCESSES AFFECTED
BY TABLE 5 TRADITIONAL MEDICINES

| | |
|---|---|
| Shc-EGFR complex (Intra-Dependence | AP-type membrane coat |
| of Viruses and the Holobiont | adaptor complex |
| https://doi.org/10.3389/fimmu.2017.01501) | |
| HOPS complex (Roy, D., Sin, S-H., | very-low-density |
| Damania, B. and Dittmer, D. P. (2011). | lipoprotein particle |
| Tumor suppressor genes FHIT and | |
| WWOX are deleted in primary effusion | |
| lymphoma (PEL) cell lines. Blood 118, | |
| e32-e39.) | |
| cytoplasmic side of early endosome | extrinsic component of |
| membrane | organelle membrane |
| GPI-anchor transamidase complex | mitochondrial proton- |
| | transporting ATP synthase |
| | complex |
| ripoptosome | proteasome regulatory |
| | particle |
| retromer, tubulation complex | lipid droplet |
| chaperonin-containing T-complex | SNARE complex |
| zona pellucida receptor complex | rough endoplasmic reticulum |
| | membrane |
| death-inducing signaling complex | melanosome |
| CD95 death-inducing signaling complex | phagocytic vesicle |
| | membrane |
| AP-2 adaptor complex | tethering complex |
| AP-3 adaptor complex | early endosome membrane |
| phagophore assembly site | nuclear euchromatin |
| flotillin complex | caveola |
| low-density lipoprotein particle | clathrin-coated vesicle |
| | membrane |
| endolysosome membrane | clathrin-coated vesicle |
| endolysosome | coated vesicle |
| tricarboxylic acid cycle enzyme complex | endosome membrane |
| microvillus membrane | early endosome |
| autophagosome membrane | late endosome |
| phagophore assembly site membrane | endocytic vesicle |
| endosome lumen | endosome |
| clathrin-coated endocytic vesicle | vacuole |
| membrane | |
| lamellar body | lysosome |
| glial cell projection | Golgi membrane |
| autophagosome | |

For identifying substances capable of supporting anti-infective properties of niclosamide and table 5 herbal medicines against pathogens identified in table 1 the methodology that can be used as described in U.S. Pat. No. 11,120,346, 27, the 717 network fragments constituting the 717P146TNTW descriptor set and a collection of over 32000 substances including prescription drugs, natural products, herbs and herbal medicines for determining information density associations in >25 million Medline abstracts. Using the methodology described in U.S. Pat. No. 11,120,346, 27 and as determinants the names of the 32000 substances and the names of network nodes constituting network fragments of the 717P146TNTW descriptor set can provide a similarity matrix which, upon hierarchical clustering, allowed identification of substances (shown in table 8) targeting protein network fragments in the 717P146TNTW descriptor set affected by niclosamide, its therapeutic equivalents and table 5 herbal medicines.

TABLE 8
4-hydroxy-2-nonenal, 5-Amino Levulinic Acid, 7-Ketocholesterol, Abemaciclib,
Abiraterone, Acetaminophen, acetylcholine, Adavosertib, Afatinib, Alectinib,
Alisertib, Alpelisib, Amlodipine, Amprenavir, Anisomycin, Aphidicolin, Arecolin,
Artesunate, Aspirn, Astaxanthin, Auranofin, Axitinib, Baicalin, Berberine,
Bermoprofen, Bevacizumab, Bexarotene, Bosentan, Bosutinib, Bromodomain
Inhibitors, Bromodomain inhibitor JQ1, Buparlisib, Caduet, Caffeine, calcitriol,
Candesartan, celastrol, Celecoxib, Ceritinib, Chloramphenicol, Cholecalciferol, CI-
1040, Ciglitazone, Cilostazol, Clarithromycin, Colchicine, Copanlisib, Costunolide,
Dabrafenib, Dacomitinib, Dicumarol, Dileucine methyl ester, Di-Leucine, Disulfiram,
Dizocilpine, Docosahexaenoic Acid, Eicosapentaenoic acid, Eicosapentaenoic acid
ethyl ester, Doxazosine, Duvelisib, Emodin, Enoxolone, Entinostat, Enzalutamide,
Enzastaurin, Epoprostenol, Epoxyeicosatrienoic acid, Eribulin, Erlotinib,
Evodiamine, Exemestane, Fasudil, Fedratinib, Fenofibrate, Fingolimod, Fluoxetine,
Gedatolisib, Geldanamycin, Genistein, Givinostat, haloperidol, Hernandezine,
Herring Roe Oil, Hymecromone, Icaritin, Icotinib, Idelalisib, Ilomastat, Imatinib,
Indomethacin, irinotecan, Ixabepilone, Kaempferol, Krill Oil, Lapatinib,
Lenalidomide, Lenvatinib, Letrozole, Liothyronine, Losartan, Lovastatin,
Luminespib, LY294002, Medroxyprogesterone, Melatonin, Menadione, Metformin,
Methotrexate, Myoinositol, Nebivolol, Nilotinib, Nimbolide, Niraparib, Obatoclax,
Olaparib, Omeprazole, Ondansetron, Orlistat, Osimertinib, Osthol, oxonic acid,
Palbociclib, Panobinostat, PD-0325901, Pemetrexed, Perifosine, Phenformin,
Phenobarbital, piperine, plicamycin, Plitidepsin, Ponicidin, Pracinostat, Pristimerin,
profolol, propofol, Pterostilbene, Puromycin, quercetin, Quinacrine, Raloxifene,
Resveratrol, Retinal, Rhein, Ribociclib, Rosiglitazone, Rosuvastatin, Ruxolitinib,
Salinomycine, Salvianolic acid, Saracatinib, Selumetinib, Semaxanib, Sildenafil,
Simvastatin, SNX-2112, Sorafenib, SP600125, Sphingosine, STAT3 Inhibitor S3I-
201, Suldinac, Sulforophane, Sunitinib, Tamoxifen, Tamsulosin, Taselisib,
Telmisartan, Teprenone, Tetracycline, Tetrahydrocurcumin, Tetrandrine,
Thalidomide, Thymoquinone, Tipifarnib, Transretinoicacid, Triamcinolone,
Trichostatin A, Troglitazone, Trolox, Tyrphostin, Umbralisib, Ursolicacid, Veliparib,
Venetoclax, Verapamil, Vinorelbine, Vitamin B12, Vorinostat, Vytorin, Withaferin A,
zinc acetate, zinc citrate, zinc gluconate, zinc pantothenate, Zinc sulfate, Zinc oxide,
Zinc chloride, Zinc phosphate.

Embodiments of the present disclosure include pharmaceutical compositions and combinations, comprising at least one compound selected from a first group of substances consisting of Niclosamide, Atovaquone, Posaconazole, Nocodazole, Nitazoxanide, JACOM Formulation, Kabasura Kudineer Chooranam preparations, Sura Kudineer preparations, Da Yuan Yin preparations, Lian Hua Qing Wen Capsule preparations, Ma Xin Gan Shi Tang preparations, Shuang Huang Lian preparations, Yin Qiao San preparations, Yu Ping Feng San preparation and combinations thereof; and one or more compounds selected from a second group consisting of 4-hydroxy-2-nonenal, 5-Amino Levulinic Acid, 7-Ketocholesterol, Abemaciclib, Abiraterone, Acetaminophen, acetylcholine, Adavosertib, Afatinib, Alectinib, Alisertib, Alpelisib, Amlodipine, Amprenavir, Anisomycin, Aphidicolin, Arecolin, Artesunate, Aspirn, Astaxanthin, Auranofin, Axitinib, Baicalin, Berberine, Bermoprofen, Bevacizumab, Bexarotene, Bosentan, Bosutinib, Bromodomain Inhibitors, Bromodomain inhibitor JQ1, Buparlisib, Caduet, Caffeine, calcitriol, Candesartan, celastrol, Celecoxib, Ceritinib, Chloramphenicol, Cholecalciferol, CI-1040, Ciglitazone, Cilostazol, Clarithromycin, Colchicine, Copanlisib, Costunolide, Dabrafenib, Dacomitinib, Dicumarol, Dileucine methyl ester, Di-Leucine, Disulfiram, Dizocilpine, Docosahexaenoic Acid, Eicosapentaenoic acid, Eicosapentaenoic acid ethyl ester, Doxazosine, Duvelisib, Emodin, Enoxolone, Entinostat, Enzalutamide, Enzastaurin, Epoprostenol, Epoxyeicosatrienoic acid, Eribulin, Erlotinib, Evodiamine, Exemestane, Fasudil, Fedratinib, Fenofibrate, Fingolimod, Fluoxetine, Gedatolisib, Geldanamycin, Genistein, Givinostat, haloperidol, Hernandezine, Herring Roe Oil, Hymecromone, Icaritin, Icotinib, Idelalisib, Ilomastat, Imatinib, Indomethacin, irinotecan, Ixabepilone, Kaempferol, Krill Oil, Lapatinib, Lenalidomide, Lenvatinib, Letrozole, Liothyronine, Losartan, Lovastatin, Luminespib, LY294002, Medroxyprogesterone, Melatonin, Menadione, Metformin, Methotrexate, Myoinositol, Nebivolol, Nilotinib, Nimbolide, Niraparib, Obatoclax, Olaparib, Omeprazole, Ondansetron, Orlistat, Osimertinib, Osthol, oxonic acid, Palbociclib, Panobinostat, PD-0325901, Pemetrexed, Perifosine, Phenformin, Phenobarbital, piperine, plicamycin, Plitidepsin, Ponicidin, Pracinostat, Pristimerin, profolol, propofol, Pterostilbene, Puromycin, quercetin, Quinacrine, Raloxifene, Resveratrol, Retinal, Rhein, Ribociclib, Rosiglitazone, Rosuvastatin, Ruxolitinib, Salinomycine, Salvianolic acid, Saracatinib, Selumetinib, Semaxanib, Sildenafil, Simvastatin, SNX-2112, Sorafenib, SP600125, Sphingosine, STAT3 Inhibitor S3I-201, Suldinac, Sulforophane, Sunitinib, Tamoxifen, Tamsulosin, Taselisib, Telmisartan, Teprenone, Tetracycline, Tetrahydrocurcumin, Tetrandrine, Thalidomide, Thymoquinone, Tipifarnib, Transretinoicacid, Triamcinolone, Trichostatin A, Troglitazone, Trolox, Tyrphostin, Umbralisib, Ursolicacid, Veliparib, Venetoclax, Verapamil, Vinorelbine, Vitamin B12, Vorinostat, Vytorin, Withaferin A, pharmaceutically acceptable zinc salts, zinc acetate, zinc citrate, zinc gluconate, zinc pantothenate, Zinc sulfate, Zinc oxide, Zinc chloride, Zinc phosphate, and a pharmaceutically acceptable zinc complexes, the pharmaceutical compositions also including a pharmaceutically acceptable carrier.

A method of using the pharmaceutical compositions or combinations, comprising an effective amount of at least one compound selected from a first group of substances consisting of Niclosamide, Atovaquone, Posaconazole, Nocodazole, Nitazoxanide, JACOM Formulation, Kabasura Kudineer Chooranam preparations, Sura Kudineer preparations, Da Yuan Yin preparations, Lian Hua Qing Wen Capsule preparations, Ma Xin Gan Shi Tang preparations, Shuang Huang Lian preparations, Yin Qiao San preparations, Yu Ping Feng San preparation and combinations thereof; and an effective amount of one or more compounds selected from a second group consisting of 4-hydroxy-2-nonenal, 5-Amino Levulinic Acid, 7-Ketocholesterol, Abemaciclib, Abiraterone, Acetaminophen, acetylcholine, Adavosertib, Afatinib, Alectinib, Alisertib, Alpelisib, Amlodipine, Amprenavir, Anisomycin, Aphidicolin, Arecolin, Artesunate, Aspirn, Astaxanthin, Auranofin, Axitinib, Baicalin, Berberine, Bermoprofen, Bevacizumab, Bexarotene, Bosentan, Bosutinib, Bromodomain Inhibitors, Bromodomain inhibitor JQ1, Buparlisib, Caduet, Caffeine, calcitriol, Candesartan, celastrol, Celecoxib, Ceritinib, Chloramphenicol, Cholecalciferol, CI-1040, Ciglitazone, Cilostazol, Clarithromycin, Colchicine, Copanlisib, Costunolide, Dabrafenib, Dacomitinib, Dicumarol, Dileucine methyl ester, Di-Leucine, Disulfiram, Dizocilpine, Docosahexaenoic Acid, Eicosapentaenoic acid, Eicosapentaenoic acid ethyl ester, Doxazosine, Duvelisib, Emodin, Enoxolone, Entinostat, Enzalutamide, Enzastaurin, Epoprostenol, Epoxyeicosatrienoic acid, Eribulin, Erlotinib, Evodiamine, Exemestane, Fasudil, Fedratinib, Fenofibrate, Fingolimod, Fluoxetine, Gedatolisib, Geldanamycin, Genistein, Givinostat, haloperidol, Hernandezine, Herring Roe Oil, Hymecromone, Icaritin, Icotinib, Idelalisib, Ilomastat, Imatinib, Indomethacin, irinotecan, Ixabepilone, Kaempferol, Krill Oil, Lapatinib, Lenalidomide, Lenvatinib, Letrozole, Liothyronine, Losartan, Lovastatin, Luminespib, LY294002, Medroxyprogesterone, Melatonin, Menadione, Metformin, Methotrexate, Myoinositol, Nebivolol, Nilotinib, Nimbolide, Niraparib, Obatoclax, Olaparib, Omeprazole, Ondansetron, Orlistat, Osimertinib, Osthol, oxonic acid, Palbociclib, Panobinostat, PD-0325901, Pemetrexed, Perifosine, Phenformin, Phenobarbital, piperine, plicamycin, Plitidepsin, Ponicidin, Pracinostat, Pristimerin, profolol, propofol, Pterostilbene, Puromycin, quercetin, Quinacrine, Raloxifene, Resveratrol, Retinal, Rhein, Ribociclib, Rosiglitazone, Rosuvastatin, Ruxolitinib, Salinomycine, Salvianolic acid, Saracatinib, Selumetinib, Semaxanib, Sildenafil, Simvastatin, SNX-2112, Sorafenib, SP600125, Sphingosine, STAT3 Inhibitor S31-201, Suldinac, Sulforophane, Sunitinib, Tamoxifen, Tamsulosin, Taselisib, Telmisartan, Teprenone, Tetracycline, Tetrahydrocurcumin, Tetrandrine, Thalidomide, Thymoquinone, Tipifarnib, Transretinoicacid, Triamcinolone, Trichostatin A, Troglitazone, Trolox, Tyrphostin, Umbralisib, Ursolicacid, Veliparib, Venetoclax, Verapamil, Vinorelbine, Vitamin B12, Vorinostat, Vytorin, Withaferin A, pharmaceutically acceptable zinc salts, zinc acetate, zinc citrate, zinc gluconate, zinc pantothenate, Zinc sulfate, Zinc oxide, Zinc chloride, Zinc phosphate, and a pharmaceutically acceptable zinc complexes (the pharmaceutical compositions also including a pharmaceutically acceptable carrier) for treatment or preventing infections in a mammal caused by pathogens selected from the group consisting of Bacterium Tuberculosis, Influenza Virus H7N2, Borna Disease Viruses, Body-1, Bodv-2, Influenza Virus H9N2, Chagas Disease, American Trypanosomiasis, Kaposi's Sarcoma-Associated Herpes virus, Human Coronavirus 229E, Hcov-229E, Lassa Virus, Crimean-Congo Hemorrhagic Fever, Leishmaniasis, Dengue Virus, Malaria, Ebola Virus, Marburg Virus, Endogenous Retroviruses, Measles, Epstein-Barr Virus, Nipah Virus, Escherichia Coli, Pertussis, Filovirus, Prion Diseases, Helicobacter Pylori, Respiratory Syncytial Virus, Hendra Henipavirus, Rift Valley Fever, Phlebovirus, Henipaviral Diseases, Salmonella, Hepatitis B Virus, Severe Acute Respiratory Syndrome Virus, Hepatitis C Virus, Shigellosis, Herpes Simplex Virus, Toxoplasmosis, HTLV-I Virus, Human Metapneumovirus, West Nile Virus, Human Papillomavirus Virus, Zika Virus, Influenza A Virus, Severe Acute Respiratory Syndrome Coronavirus SARS-Cov-2, Severe Acute Respiratory Syndrome Coronavirus SARS-Cov-1, Middle East Respiratory Syndrome Virus MERS, Trichophyton, Microsporum, Epidermophyton species, Candida, Aspergillus, Cryptococcus, and Pneumocystis.

Embodiments of the present disclosure include pharmaceutical compositions and combinations, comprising niclosamide and at least one compound selected from a second group of compounds is selected from the group consisting of bexarotene, Celecoxib, PD184352, Ciglitazone, Evodiamine, Fingolimod, geldanamycin, Obatoclax, Ondansetron, Perifosine, Phenformin, Ponicidin, Raloxifene, Sorafenib, and Troglitazone, the pharmaceutical compositions also including a pharmaceutically acceptable carrier.

Embodiments of the present disclosure include methods of using a using the pharmaceutical compositions or combination including niclosamide and at least one compound selected from a second group of compounds is selected from the group consisting of bexarotene, Celecoxib, PD184352, Ciglitazone, Evodiamine, Fingolimod, geldanamycin, Obatoclax, Ondansetron, Perifosine, Phenformin, Ponicidin, Raloxifene, Sorafenib, and Troglitazone (the pharmaceutical compositions also including a pharmaceutically acceptable carrier) for treatment of cancer selected from the group consisting of transitional cell carcinoma of the urinary bladder, bladder cancer, breast carcinoma, breast cancer, Chronic Lymphoblastic Leukemia. colorectal carcinoma, esophageal cancer, gastric cancer, head and neck carcinoma, lymph node metastasis, lymphoma, meningioma, Metastatic Breast Cancer, metastatic colorectal cancer, metastatic lung adenocarcinoma, non-small cell lung cancer with acquired resistance to EGFR-TKIs, oral leukoplakias and oral squamous cell carcinomas, ovarian cancer, Pseudomesotheliomatous Carcinoma, and squamous cell carcinoma of the cervix Urothelial cancer.

Embodiments of the present disclosure include pharmaceutical compositions and combinations, comprising at least one compound selected from a first group of substances consisting of Docosahexaenoic Acid, Eicosapentaenoic acid, Krill oil, Herring roe oil, Eicosapentaenoic acid ethyl ester, Eicosatrienoic acid, Eicosatrienoic acid ethyl ester, cannabidiol, hemp oil and combinations thereof and one or more compounds selected from a second group of substances consisting of Myo-inositol, Dileucine, mung bean protein, Krill protein, Herring roe protein, Pterostilbene, and Caffeine, the pharmaceutical compositions also including a pharmaceutically acceptable carrier.

Embodiments of the present disclosure include methods of using the pharmaceutical compositions or combination including at least one compound selected from a first group of substances consisting of Docosahexaenoic Acid, Eicosapentaenoic acid, Krill oil, Herring roe oil, Eicosapentaenoic acid ethyl ester, Eicosatrienoic acid, Eicosatrienoic acid ethyl ester, cannabidiol, hemp oil and combinations thereof and one or more compounds selected from a second group of substances consisting of Myo-inositol, Dileucine, mung bean protein, Krill protein, Herring roe protein, Pterostilbene, and Caffeine (the pharmaceutical compositions also including a pharmaceutically acceptable carrier) for treatment or preventing infections and associated symptoms caused by pathogens selected from the group consisting of Bacterium Tuberculosis, Influenza Virus H7N2, Borna Disease Viruses, Body-1, Bodv-2, Influenza Virus H9N2, Chagas Disease, American Trypanosomiasis, Kaposi's Sarcoma-Associated Herpes virus, Human Coronavirus 229E, Hcov-229E, Lassa Virus, Crimean-Congo Hemorrhagic Fever, Leishmaniasis, Dengue Virus, Malaria, Ebola Virus, Marburg Virus, Endogenous Retroviruses, Measles, Epstein-Barr Virus, Nipah Virus, Escherichia Coli, Pertussis, Filovirus, Prion Diseases, Helicobacter Pylori, Respiratory Syncytial Virus, Hendra Henipavirus, Rift Valley Fever, Phlebovirus, Henipaviral Diseases, Salmonella, Hepatitis B Virus, Severe Acute Respiratory Syndrome Virus, Hepatitis C Virus, Shigellosis, Herpes Simplex Virus, Toxoplasmosis, HTLV-I Virus, Human Metapneumovirus, West Nile Virus, Human Papillomavirus Virus, Zika Virus, Influenza A Virus, Severe Acute Respiratory Syndrome Coronavirus SARS-Cov-2, Severe Acute Respiratory Syndrome Coronavirus SARS-Cov-1, Middle East Respiratory Syndrome Virus MERS, Trichophyton, Microsporum, Epidermophyton species, Candida, Aspergillus, Cryptococcus, and Pneumocystis.

Embodiments of the present disclosure include a biological structure-function constraint topological descriptor set termed 11KTSPDS.

Embodiments of the present disclosure include a method for constructing descriptor set 11KTSPDS comprising a first step selecting tissue specific expression data of protein encoding genes, a second step using said selected genes for construction protein-protein interaction networks termed primary networks, a third step using gene enrichment analysis for identifying protein network nodes of said primary protein protein interaction networks that co-occur in gene ontology based biological process networks termed secondary networks providing protein network fragments creating protein network overlaps between said primary and secondary networks, a fourth step of collecting said protein network fragments in an intermittent database, a fifth step of using said collected protein network fragments for selecting network fragments containing no more than ten network nodes associated with a false discovery rate of at least 0.001, a sixth step for associating said selected protein network fragments with registration codes identifying biological process network and tissue network of origin and a seventh step of collecting eleven thousand one hundred of said registration code associated protein network fragments providing descriptor set 11KTSPDS.

Embodiments of the present disclosure include a method of using descriptor set 11KTSPDS in biological structure function analysis.

Embodiments of the present disclosure include a biological structure-function constraint topological descriptor set termed PATHPPI.

Embodiments of the present disclosure include a method for creating descriptor set PATHPPI comprising a first step selecting protein encoding genes associated with infections caused by Bacterium Tuberculosis, Influenza Virus H7N2, Borna Disease Viruses, Body-1, Bodv-2, Influenza Virus H9N2, Chagas Disease, American Trypanosomiasis, Kaposi's Sarcoma-Associated Herpes virus, Human Coronavirus 229E, Hcov-229E, Lassa Virus, Crimean-Congo Hemorrhagic Fever, Leishmaniasis, Dengue Virus, Malaria, Ebola Virus, Marburg Virus, Endogenous Retroviruses, Measles, Epstein-Barr Virus, Nipah Virus, Escherichia Coli, Pertussis, Filovirus, Prion Diseases, Helicobacter Pylori, Respiratory Syncytial Virus, Hendra Henipavirus, Rift Valley Fever, Phlebovirus, Henipaviral Diseases, Salmonella, Hepatitis B Virus, Severe Acute Respiratory Syndrome Virus, Hepatitis C Virus, Shigellosis, Herpes Simplex Virus, Toxoplasmosis, HTLV-I Virus, Human Metapneumovirus, West Nile Virus, Human Papillomavirus Virus, Zika Virus, Influenza A Virus, Severe Acute Respiratory Syndrome Coronavirus SARS-Cov-2, Severe Acute Respiratory Syndrome Coronavirus SARS-Cov-1, Middle East Respiratory Syndrome Virus MERS, Trichophyton, Microsporum, Epidermophyton species, Candida, Aspergillus, Cryptococcus, or Pneumocystis, a second step using said selected genes for construction protein-protein interaction networks termed primary protein interaction networks, a third step using the descriptor set of claim 26 for identifying protein network fragments containing protein network nodes that co-occur in said primary protein interaction networks and said descriptor set of claim 26, a fourth step of collecting and storing said protein network fragments in an intermittent database, a fifth step of identifying protein network nodes of said collected protein network fragments, a sixth step using said protein network nodes for constructing an intermittent protein protein interaction network, a seventh step using said intermittent protein interaction network and gene enrichment analysis for identifying protein network fragments containing network nodes that occur in said intermittent protein protein interaction network and gene ontology based biological processes protein interaction networks and an eight step of collecting said protein network fragments a nineth step using said network fragment collections for selecting network fragments containing no more than thirty network nodes associated with a false discovery rate of at least 0.001 for providing descriptor set PATHPPI.

Embodiments of the present disclosure include a method of using descriptor set PATHPPI for identifying substances for treatment or preventions of infections caused by Bacterium Tuberculosis, Influenza Virus H7N2, Borna Disease Viruses, Body-1, Body-2, Influenza Virus H9N2, Chagas Disease, American Trypanosomiasis, Kaposi's Sarcoma-Associated Herpes virus, Human Coronavirus 229E, Hcov-229E, Lassa Virus, Crimean-Congo Hemorrhagic Fever, Leishmaniasis, Dengue Virus, Malaria, Ebola Virus, Marburg Virus, Endogenous Retroviruses, Measles, Epstein-Barr Virus, Nipah Virus, Escherichia Coli, Pertussis, Filovirus, Prion Diseases, Helicobacter Pylori, Respiratory Syncytial Virus, Hendra Henipavirus, Rift Valley Fever, Phlebovirus, Henipaviral Diseases, Salmonella, Hepatitis B Virus, Severe Acute Respiratory Syndrome Virus, Hepatitis C Virus, Shigellosis, Herpes Simplex Virus, Toxoplasmosis, HTLV-I Virus, Human Metapneumovirus, West Nile Virus, Human Papillomavirus Virus, Zika Virus, Influenza A Virus, Severe Acute Respiratory Syndrome Coronavirus SARS-Cov-2, Severe Acute Respiratory Syndrome Coronavirus SARS-Cov-1, Middle East Respiratory Syndrome Virus MERS, Trichophyton, Microsporum, Epidermophyton species, Candida, Aspergillus, Cryptococcus, or Pneumocystis.

Embodiments of the present disclosure include a method of using descriptor set PATHPPI in biological structure function analysis.

Embodiments of the present disclosure include a method of using descriptor set PATHPPI for identifying substances and substance combinations for treating and or preventing infections and diseases caused by a broad range of pathogens of diverse origins.

All publications, including but not limited to, issued patents, patent applications, and journal articles, cited in this application are each herein incorporated by reference in their entirety.

Thus, while there have been shown, described and pointed out, fundamental novel features of the present disclosure as applied to the exemplary embodiments thereof, it will be understood that various omissions and substitutions and changes in the form and details of devices and methods illustrated, and in their operation, may be made by those skilled in the art without departing from the spirit or scope of the present disclosure. Moreover, it is expressly intended that all combinations of those elements and/or method steps, which perform substantially the same function in substantially the same way to achieve the same results, are within the scope of the present disclosure. Moreover, it should be recognized that structures and/or elements and/or method steps shown and/or described in connection with any disclosed form or embodiment of the present disclosure may be incorporated in any other disclosed or described or suggested form or embodiment as a general matter of design choice. It is the intention, therefore, to be limited only as indicated by the scope of the claims appended hereto.

This written description uses examples as part of the disclosure, including the best mode, and also to enable any person skilled in the art to practice the disclosed implementations, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

While there have been shown, described and pointed out, fundamental features of the present disclosure as applied to the exemplary embodiments thereof, it will be understood that various omissions and substitutions and changes in the form and details of compositions, devices and methods illustrated, and in their operation, may be made by those skilled in the art without departing from the spirit or scope of the present disclosure. Moreover, it is expressly intended that all combinations of those elements and/or method steps, which perform substantially the same function in substantially the same way to achieve the same results, are within the scope of the present disclosure. Moreover, it should be recognized that structures and/or elements and/or method steps shown and/or described in connection with any disclosed form or embodiment of the present disclosure may be incorporated in any other disclosed or described or suggested form or embodiment as a general matter of design choice. It is the intention, therefore, to be limited only as indicated by the scope of the claims appended hereto.

1. A combination of substances comprising at least one compound selected from a first group of substances consisting of Niclosamide, Atovaquone, Posaconazole, Nocodazole, Nitazoxanide, JACOM Formulation, Kabasura Kudineer Chooranam preparations, Sura Kudineer preparations, Da Yuan Yin preparations, Lian Hua Qing Wen Capsule preparations, Ma Xin Gan Shi Tang preparations, Shuang Huang Lian preparations, Yin Qiao San preparations, Yu Ping Feng San preparation and combinations thereof and one or more compounds selected from a second group consisting of 4-hydroxy-2-nonenal, 5-Amino Levulinic Acid, 7-Ketocholesterol, Abemaciclib, Abiraterone, Acetaminophen, acetylcholine, Adavosertib, Afatinib, Alectinib, Alisertib, Alpelisib, Amlodipine, Amprenavir, Anisomycin, Aphidicolin, Arecolin, Artesunate, Aspirin, Astaxanthin, Auranofin, Axitinib, Baicalin, Berberine, Bermoprofen, Bevacizumab, Bexarotene, Bosentan, Bosutinib, Bromodomain Inhibitors, Bromodomain inhibitor JQ1, Buparlisib, Caduet, Caffeine, calcitriol, Candesartan, celastrol, Celecoxib, Ceritinib, Chloramphenicol, Cholecalciferol, CI-1040, Ciglitazone, Cilostazol, Clarithromycin, Colchicine, Copanlisib, Costunolide, Dabrafenib, Dacomitinib, Dicumarol, Dileucine methyl ester, Di-Leucine, Disulfiram, Dizocilpine, Docosahexaenoic Acid, Eicosapentaenoic acid, Eicosapentaenoic acid ethyl ester, Doxazosine, Duvelisib, Emodin, Enoxolone, Entinostat, Enzalutamide, Enzastaurin, Epoprostenol, Epoxyeicosatrienoic acid, Eribulin, Erlotinib, Evodiamine, Exemestane, Fasudil, Fedratinib, Fenofibrate, Fingolimod, Fluoxetine, Gedatolisib, Geldanamycin, Genistein, Givinostat, haloperidol, Hernandezine, Herring Roe Oil, Hymecromone, Icaritin, Icotinib, Idelalisib, Ilomastat, Imatinib, Indomethacin, irinotecan, Ixabepilone, Kaempferol, Krill Oil, Lapatinib, Lenalidomide, Lenvatinib, Letrozole, Liothyronine, Losartan, Lovastatin, Luminespib, LY294002, Medroxyprogesterone, Melatonin, Menadione, Metformin, Methotrexate, Myoinositol, Nebivolol, Nilotinib, Nimbolide, Niraparib, Obatoclax, Olaparib, Omeprazole, Ondansetron, Orlistat, Osimertinib, Osthol, oxonic acid, Palbociclib, Panobinostat, PD-0325901, Pemetrexed, Perifosine, Phenformin, Phenobarbital, piperine, plicamycin, Plitidepsin, Ponicidin, Pracinostat, Pristimerin, profolol, propofol, Pterostilbene, Puromycin, quercetin, Quinacrine, Raloxifene, Resveratrol, Retinal, Rhein, Ribociclib, Rosiglitazone, Rosuvastatin, Ruxolitinib, Salinomycine, Salvianolic acid, Saracatinib, Selumetinib, Semaxanib, Sildenafil, Simvastatin, SNX-2112, Sorafenib, SP600125, Sphingosine, STAT3 Inhibitor S31-201, Suldinac, Sulforophane, Sunitinib, Tamoxifen, Tamsulosin, Taselisib, Telmisartan, Teprenone, Tetracycline, Tetrahydrocurcum in, Tetrandrine, Thalidomide, Thymoquinone, Tipifarnib, Transretinoicacid, Triamcinolone, Trichostatin A, Troglitazone, Trolox, Tyrphostin, Umbralisib, Ursolicacid, Veliparib, Venetoclax, Verapamil, Vinorelbine, Vitamin B12, Vorinostat, Vytorin, Withaferin A, pharmaceutically acceptable zinc salts, zinc acetate, zinc citrate, zinc gluconate, zinc pantothenate, Zinc sulfate, Zinc oxide, Zinc chloride, Zinc phosphate, and a pharmaceutically acceptable zinc complexes.

2. The combination of substances according to claim 1, wherein the at least one compound selected from said first group of substances includes Niclosamide and the one or more compounds selected from said second group are selected from the group consisting of Entinostat, Caduet, phenformin, quinacrine, Vytorin, Panobinostat, tamsulosin, myoinositol, pterostilbene, omeprazole, Retinal, pharmaceutically acceptable zinc salts, and pharmaceutically acceptable zinc complexes.

3. The combination of substances according to claim 1, wherein the at least one compound selected from said first group of substances are selected from the group consisting of Atovaquone, Posaconazole, Nocodazole, and Nitazoxanide.

4. The combination of substances according to claim 1, wherein the one or more compounds selected from the second group of compounds are selected from the group consisting of Entinostat, Caduet, phenformin, quinacrine, Vytorin, Panobinostat, tamsulosin, myoinositol, pterostilbene, omeprazole, Retinal, pharmaceutically acceptable zinc salts, and a pharmaceutically acceptable zinc complexes.

5. The combination of substances according to claim 4, wherein the at least one compound selected from said first group of substances is Atovaquone.

6. The combination of substances according to claim 4, wherein the at least one compound selected from said first group of substances is Posaconazole.

7. The combination of substances according to claim 4, wherein the at least one compound selected from said first group of substances is Nocodazole.

8. The combination of substances according to claim 4, wherein the at least one compound selected from said first group of substances is Nitazoxanide.

9. The combination of substances according to claim 1, wherein the one or more compounds selected from said second group is colchicine.

10. The combination of substances according to claim 1, wherein the one or more compounds selected from said second group is Entinostat.

11. The combination of substances according to claim 1, wherein the one or more compounds selected from said second group is phenformin.

12. The combination of substances according to claim 5, wherein the one or more compounds selected from said second group is selected from the group consisting of zinc gluconate, zinc acetate, zinc pantothenate, zinc oxide, zinc chloride, zinc sulfate, zinc phosphate, a pharmaceutically acceptable zinc salt, and a pharmaceutically acceptable zinc complex.

13. The combination of substances according to claim 5, wherein the one or more compounds selected from said second group is myoinositol.

14. The combination of substances according to claim 1, wherein the one or more compounds selected from said second group is trans retinoic acid.

15. The combination of substances according to claim 1, wherein the one or more compounds selected from said second group is Disulfiram.

16. The combination of substances according to claim 1, wherein the one or more compounds selected from said second group is selected from the group consisting of dileucine, and dileucine methyl ester.

17. A method for treatment or preventing infections in a mammal caused by pathogens selected from the group consisting of Bacterium Tuberculosis, Influenza Virus H7N2, Borna Disease Viruses, Body-1, Bodv-2, Influenza Virus H9N2, Chagas Disease, American Trypanosomiasis, Kaposi's Sarcoma-Associated Herpes virus, Human Coronavirus 229E, Hcov-229E, Lassa Virus, Crimean-Congo Hemorrhagic Fever, Leishmaniasis, Dengue Virus, Malaria, Ebola Virus, Marburg Virus, Endogenous Retroviruses, Measles, Epstein-Barr Virus, Nipah Virus, Escherichia Coli, Pertussis, Filovirus, Prion Diseases, Helicobacter Pylori, Respiratory Syncytial Virus, Hendra Henipavirus, Rift Valley Fever, Phlebovirus, Henipaviral Diseases, Salmonella, Hepatitis B Virus, Severe Acute Respiratory Syndrome Virus, Hepatitis C Virus, Shigellosis, Herpes Simplex Virus, Toxoplasmosis, HTLV-I Virus, Human Metapneumovirus, West Nile Virus, Human Papillomavirus Virus, Zika Virus, Influenza A Virus, Severe Acute Respiratory Syndrome Coronavirus SARS-Cov-2, Severe Acute Respiratory Syndrome Coronavirus SARS-Cov-1, Middle East Respiratory Syndrome Virus MERS, Trichophyton, Microsporum, Epidermophyton species, Candida, Aspergillus, Cryptococcus, and Pneumocystis, the method comprising administering to said mammal in need of such treatment or prevention an effective amount of at least one compound selected from a first group of substances consisting of Niclosamide, Atovaquone, Posaconazole, Nocodazole, Nitazoxanide, JACOM Formulation, Kabasura Kudineer Chooranam preparations, Sura Kudineer preparations, Da Yuan Yin preparations, Lian Hua Qing Wen Capsule preparations, Ma Xin Gan Shi Tang preparations, Shuang Huang Lian preparations, Yin Qiao San preparations, Yu Ping Feng San preparation and combinations thereof and an effective amount of one or more compounds selected from a second group consisting of 4-hydroxy-2-nonenal, 5-Amino Levulinic Acid, 7-Ketocholesterol, Abemaciclib, Abiraterone, Acetaminophen, acetylcholine, Adavosertib, Afatinib, Alectinib, Alisertib, Alpelisib, Amlodipine, Amprenavir, Anisomycin, Aphidicolin, Arecolin, Artesunate, Aspirin, Astaxanthin, Auranofin, Axitinib, Baicalin, Berberine, Bermoprofen, Bevacizumab, Bexarotene, Bosentan, Bosutinib, Bromodomain Inhibitors, Bromodomain inhibitor JQ1, Buparlisib, Caduet, Caffeine, calcitriol, Candesartan, celastrol, Celecoxib, Ceritinib, Chloramphenicol, Cholecalciferol, CI-1040, Ciglitazone, Cilostazol, Clarithromycin, Colchicine, Copanlisib, Costunolide, Dabrafenib, Dacomitinib, Dicumarol, Dileucine methyl ester, Di-Leucine, Disulfiram, Dizocilpine, Docosahexaenoic Acid, Eicosapentaenoic acid, Eicosapentaenoic acid ethyl ester, Doxazosine, Duvelisib, Emodin, Enoxolone, Entinostat, Enzalutamide, Enzastaurin, Epoprostenol, Epoxyeicosatrienoic acid, Eribulin, Erlotinib, Evodiamine, Exemestane, Fasudil, Fedratinib, Fenofibrate, Fingolimod, Fluoxetine, Gedatolisib, Geldanamycin, Genistein, Givinostat, haloperidol, Hernandezine, Herring Roe Oil, Hymecromone, Icaritin, Icotinib, Idelalisib, Ilomastat, Imatinib, Indomethacin, irinotecan, Ixabepilone, Kaempferol, Krill Oil, Lapatinib, Lenalidomide, Lenvatinib, Letrozole, Liothyronine, Losartan, Lovastatin, Luminespib, LY294002, Medroxyprogesterone, Melatonin, Menadione, Metformin, Methotrexate, Myoinositol, Nebivolol, Nilotinib, Nimbolide, Niraparib, Obatoclax, Olaparib, Omeprazole, Ondansetron, Orlistat, Osimertinib, Osthol, oxonic acid, Palbociclib, Panobinostat, PD-0325901, Pemetrexed, Perifosine, Phenformin, Phenobarbital, piperine, plicamycin, Plitidepsin, Ponicidin, Pracinostat, Pristimerin, profolol, propofol, Pterostilbene, Puromycin, quercetin, Quinacrine, Raloxifene, Resveratrol, Retinal, Rhein, Ribociclib, Rosiglitazone, Rosuvastatin, Ruxolitinib, Salinomycine, Salvianolic acid, Saracatinib, Selumetinib, Semaxanib, Sildenafil, Simvastatin, SNX-2112, Sorafenib, SP600125, Sphingosine, STAT3 Inhibitor S31-201, Suldinac, Sulforophane, Sunitinib, Tamoxifen, Tamsulosin, Taselisib, Telmisartan, Teprenone, Tetracycline, Tetrahydrocurcum in, Tetrandrine, Thalidomide, Thymoquinone, Tipifarnib, Transretinoicacid, Triamcinolone, Trichostatin A, Troglitazone, Trolox, Tyrphostin, Umbralisib, Ursolicacid, Veliparib, Venetoclax, Verapamil, Vinorelbine, Vitamin B12, Vorinostat, Vytorin, Withaferin A, pharmaceutically acceptable zinc salts, zinc acetate, zinc citrate, zinc gluconate, zinc pantothenate, Zinc sulfate, Zinc oxide, Zinc chloride, Zinc phosphate, and a pharmaceutically acceptable zinc complexes.

18. A combination of substances comprising niclosamide and one or more compounds selected from a second group consisting of bexarotene, Celecoxib, PD184352, Ciglitazone, Evodiamine, Fingolimod, geldanamycin, Obatoclax, Ondansetron, Perifosine, Phenformin, Ponicidin, Raloxifene, Sorafenib, and Troglitazone.

19. A combination of substances comprising at least one compound selected from a first group of substances consisting of Docosahexaenoic Acid, Eicosapentaenoic acid, Krill oil, Herring roe oil, Eicosapentaenoic acid ethyl ester, Eicosatrienoic acid, Eicosatrienoic acid ethyl ester, cannabidiol, hemp oil and combinations thereof and one or more compounds selected from a second group consisting of Myo-inositol, Dileucine, mung bean protein, Krill protein, Herring roe protein, Pterostilbene, and Caffeine.

20. A method of using the combination of substances to claim 19 for treatment or preventing infections and associated symptoms in a mammal caused by pathogens selected from the group consisting of Bacterium Tuberculosis, Influenza Virus H7N2, Borna Disease Viruses, Body-1, Bodv-2, Influenza Virus H9N2, Chagas Disease, American Trypanosomiasis, Kaposi's Sarcoma-Associated Herpes virus, Human Coronavirus 229E, Hcov-229E, Lassa Virus, Crimean-Congo Hemorrhagic Fever, Leishmaniasis, Dengue Virus, Malaria, Ebola Virus, Marburg Virus, Endogenous Retroviruses, Measles, Epstein-Barr Virus, Nipah Virus, Escherichia Coli, Pertussis, Filovirus, Prion Diseases, Helicobacter Pylori, Respiratory Syncytial Virus, Hendra Henipavirus, Rift Valley Fever, Phlebovirus, Henipaviral Diseases, Salmonella, Hepatitis B Virus, Severe Acute Respiratory Syndrome Virus, Hepatitis C Virus, Shigellosis, Herpes Simplex Virus, Toxoplasmosis, HTLV-I Virus, Human Metapneumovirus, West Nile Virus, Human Papillomavirus Virus, Zika Virus, Influenza A Virus, Severe Acute Respiratory Syndrome Coronavirus SARS-Cov-2, Severe Acute Respiratory Syndrome Coronavirus SARS-Cov-1, Middle East Respiratory Syndrome Virus MERS, Trichophyton, Microsporum, Epidermophyton species, Candida, Aspergillus, Cryptococcus, and Pneumocystis the method comprising administering to said mammal in need of such treatment or prevention an effective amount of at least one compound selected from a first group of substances and an effective amount of one or more compounds selected from a second group.

21. A biological structure-function constraint topological descriptor set termed 11KTSPDS.

22. A method for constructing descriptor set 11KTSPDS comprising a first step of selecting tissue specific expression data of protein encoding genes, a second step of using said selected genes for construction protein-protein interaction networks termed primary networks, a third step of using gene enrichment analysis for identifying protein network nodes of said primary protein protein interaction networks that co-occur in gene ontology based biological process networks termed secondary networks providing protein network fragments creating protein network overlaps between said primary and secondary networks, a fourth step of collecting said protein network fragments in an intermittent database, a fifth step of using said collected protein network fragments for selecting network fragments containing no more than ten network nodes associated with a false discovery rate of at least 0.001, a sixth step for associating said selected protein network fragments with registration codes identifying biological process network and tissue network of origin and a seventh step of collecting eleven thousand one hundred of said registration code associated protein network fragments providing descriptor set 11KTSPDS.

23. A method of using the descriptor set of claim 21 in biological structure function analysis.
